(19) United States
(12) Patent Application Publication
(10) Pub. No.: US 2008/0275134 A1
(43) Pub. Date: Nov. 6, 2008

(54) Methods for Treatment of Retinal Degenerative Disease

(75) Inventors: Krzysztof Palczewski, Bay Village, OH (US); Marcin Golczak, Mayfield Heights, OH (US); Vladimir Kuksa, Seattle, WA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: University of Washington, Seattle, WA (US)

(21) Appl. No.: 11/817,015

(22) PCT Filed: Feb. 24, 2006
(86) PCT No.: PCT/US2006/006507

§ 371 Date: Jun. 20, 2008

Related U.S. Application Data
(60) Provisional application No. 60/775,994, filed on Feb. 24, 2005.
(60) Provisional application No. 60/714,546, filed on Mar. 18, 2005.

(51) Int. Cl. A61K 31/07; A61P 27/02

(52) U.S. Cl. 514/725

(57) ABSTRACT

A method is provided for treating a degenerative disease in a vertebrate eye. A method is further provided for preventing photoreceptor degeneration in a vertebrate eye.

### CROSS REFERENCE TO RELATED APPLICATIONS

This application claims benefit of U.S. application Ser. No. ______, filed Feb. 24, 2005 [UW-0009], and claims benefit of U.S. application Ser. No. ______, filed Mar. 18, 2005 [UW-0014], the disclosure of which is incorporated herein by reference in its entirety.

### STATEMENT OF GOVERNMENT SUPPORT

This invention was made with Government support by Grant No. EY008061, awarded by the National Eye Institute. The Government has certain rights in this invention.

### FIELD

The present invention provides a method for treating a degenerative disease in a vertebrate eye. The present invention further provides a method for preventing photoreceptor degeneration in a vertebrate eye.

### BACKGROUND

In vertebrate photoreceptor cells, a photon causes isomerization of the 11-cis-retinylidene chromophore to all-trans-retinylidene coupled to the visual opsin receptors. This photoisomerization triggers conformational changes of opsins which, in turn, initiate the biochemical chain of reactions termed phototransduction (Filipek et al., Annu Rev Physiol 65: 851-79, 2003). The regeneration of the visual pigments requires that the chromophore be converted back to the 11-cis-configuration in the processes collectively called the retinoid visual) cycle reviewed in McBee et al., Prog Retin Eye Res 20:469-52, 2001). First, the chromophore is released from the opsin and reduced in the photoreceptor by retinol dehydrogenases. The product, all-trans-retinol, is trapped in the adjacent retinal pigment epithelium (RPE) in the form of insoluble fatty acid esters in subcellular structures known as retinosomes (Imanishi et al., J Cell Biol 164:373-8, 2004). The key isomerization process still remains elusive to molecular characterization. The “isomerohydrolase” hypothesis proposes the existence of an enzyme that would utilize the energy of retinyl ester hydrolysis to carry out the endothermic isomerization reaction (Rando, Biochemistry 30:595-602, 1990). This mechanism entails a nucleophilic attack at the C₁₁ position of all-trans-retinyl palmitate with concurrent elimination of palmitate by alkyl cleavage (FIG. 1A). The complex rotates to reposition the C₁₁-C₁₂ bond into a new conformation followed by rehydration of the transition state of the chromophore-protein complex, leading to the production of 11-cis-retinol. There is a lack of direct evidence for this mechanism, and its pros and cons have been extensively discussed (Kuksa et al., Vision Res 43:2959-81, 2003). An alternative mechanism has been proposed by our laboratory, in which all-trans-retinyl esters are converted into an unidentified intermediate (all-trans-retinol, a subpopulation of activated esters, or an unknown retinoid intermediate) (Stecher et al., J Biol Chem 274:8577-85, 1999). This intermediate is then converted to retinyl carbocation, rehydrated in the transition state, and released as 11-cis-retinol (McBee et al., Biochemistry 39:11370-80, 2000) (FIG. 1B). Significant product formation in this endothermic reaction should only be seen in the presence of retinoid-binding proteins (Stecher and Palczewski, Methods Enzymol 316:330-44, 2000), and the ratio of the isomers produced appears to be sensitive to the specificity of the retinoid-binding proteins (Stecher et al., J Biol Chem 274:8577-85, 1999; McBee et al., Biochemistry 39:11370-80, 2000). In both mechanisms the pathway would progress via an alkyl cleavage as observed experimentally (see Kuksa et al., Vision Res 43:2959-81, 2003 for summary and discussion). Based on the second mechanism, potent transition state analogs could be designed to inhibit the isomerization reaction. Positively changed retinoids would inhibit the enzyme if the reaction occurred via the carbocation intermediate, whereas the isomerohydrolase mechanism should be much less affected.

The use of potent inhibitors of isomerization in vivo after light exposure would also prevent or slow down the recovery of the visual pigment chromophore production. In Stargardt's disease (Allikmets et al., Nat Genet 15:236-46, 1997), associated with mutations in the ABCR transporter, the accumulation of all-trans-retinal has been proposed to be responsible for the formation of a lipofuscin pigment, A2E, which is toxic towards retinal cells and causes retinal degeneration and consequently loss of vision (Mata and Travis, Proc Natl Acad Sci USA 97:7154-9, 2000; Weng et al., Cell 98:13-23, 1999). It was proposed that treating patients with an inhibitor of retinol dehydrogenases, 13-cis-RA (Accutane®, Roche), might prevent or slow down the formation of A2E and might have protective properties to maintain normal vision (Radu et al., Proc Natl Acad Sci USA 100:4742-7, 2003). 13-cis-RA (Isotretinoin, or Accutane®) inhibits 11-cis-RDH (Law and Rando, Biochem Biophys Res Commun 161:825-9, 1989) and is associated with induced night blindness, has been used to slow the synthesis of 11-cis-retinal through the inhibition of 11-cis-RDH. Others have proposed that 13-cis-RA works to prevent chromophore regeneration by binding RPE65, a protein essential for the isomerization process in the eye (Gollapalli and Rando, Proc Natl Acad Sci USA 101: 10030-5, 2004; WO 2005/079774; WO 2006/007314). These investigators found that 13-cis-RA blocked the formation of A2E, and suggested that this treatment may inhibit lipofuscin accumulation and thus delay either the onset of visual loss in Stargardt's patients or the macular degeneration associated with lipofuscin accumulation. One must be aware of a potential problem associated with blocking the retinoid cycle and the formation of unliganded opsin (Van Hooser et al., J Biol Chem 277:19173-82, 2002; Woodruff et al., Nat Genet. 35:158-164, 2003). This may result in more severe consequences and worsening of the patient's prognosis. Failure of the chromophore to form may lead to progressive retinal degeneration and in an extreme case will produce phenotype similar to Leber Congenital Amaurosis (LCA). This disease is a very rare childhood condition that effects children from birth or shortly there after. Furthermore treatment with 13-cis-RA is associated with induced night blindness. A need exists in the art for an effective treatment for Stargardt's disease and age-related macular degeneration (AMD) without causing further unwanted side effects such as progressive retinal degeneration, LCA, or night blindness.

### SUMMARY OF THE INVENTION

The present invention provides a method for treating a degenerative disease in a vertebrate eye, comprising administering to the vertebrate an effective amount of a positively charged retinoid derivative e.g., a retinylamine derivative, in a pharmaceutically or ophthamologically acceptable vehicle. The degenerative disease is age-related macular degeneration or Stargardt's macular dystrophy. The present invention further provides a method for preventing photoreceptor degeneration in a vertebrate eye or a method for restoring photoreceptor function in a vertebrate eye comprising administering to the vertebrate an effective amount of a positively charged retinoid compound, e.g., a retinylamine derivative, in a pharmaceutically or ophthamologically acceptable vehicle to slow chromophore flux in a retinoid cycle in the eye and restore photoreceptor function in the eye.

A method for treatment or prophylaxis of a degenerative disease in a vertebrate eye is provided which comprises administering to the vertebrate an effective amount of a positively charged retinoid derivative in a pharmaceutically or ophthamologically acceptable vehicle. In one embodiment, the positively charged retinoid derivative is a retinylamine derivative. In a further aspect, the positively charged retinoid derivative inhibits an isomerization step of the retinoid cycle.

A method for preventing photoreceptor degeneration in a vertebrate eye or a method for restoring photoreceptor function in a vertebrate eye is provided which comprises administering to the vertebrate an effective amount of a positively charged retinoid compound in a pharmaceutically or ophthamologically acceptable vehicle, slowing chromophore flux in a retinoid cycle in the eye, and preventing photoreceptor degeneration in a vertebrate eye or a method for restoring photoreceptor function in the eye. In one embodiment, the positively charged retinoid derivative is a retinylamine derivative. In a further aspect, the positively charged retinoid derivative inhibits an isomerization step of the retinoid cycle.

### BRIEF DESCRIPTION OF THE DRAWINGS

FIGS. 1A and 1B show two proposed mechanisms of 11-cis-retinol formation.

FIGS. 2A, 2B, 2C, and 2D show inhibition of 11-cis-retinol isomerase activity by retinylamine and its derivatives.

FIGS. 3A and 3B show gel filtration chromatography of RPE proteins.

FIGS. 4A, 4B, 4C, and 4D show retinylamine inhibits regeneration of vision chromophore in vivo.

FIGS. 5A, 5B, and 5C show synthesis and HPLC separation of retinylamine isomers.

FIGS. 6A and 6B show retinylamine inhibits regeneration of vision chromophore in vivo after intense bleach.

FIGS. 7A and 7B show single flash ERG responses of increasing intensity for all-trans-retinylamine treated mice and control mice in light-adapted conditions.

FIG. 8 shows response of F9-RARE-lacZ reporter cell line to retinylamine.

FIG. 9 shows activation of DR1-elements by retinylamine. HEK-293 cells were transfected with a construct of lacZ under the control of a minimal promoter and five consecutive upstream DR1 elements.

FIG. 10 shows retinylamides inhibit regeneration of vision chromophore in mice after treatment with retinylamide.

FIG. 11 shows structures of possible prodrugs of retinylamine.

### DETAILED DESCRIPTION OF ILLUSTRATIVE EMBODIMENTS

The present invention provides a method for treating a degenerative disease in a vertebrate eye, comprising administering to the vertebrate an effective amount of a positively charged retinoid derivative e.g., a retinylamine derivative, in a pharmaceutically or ophthamologically acceptable vehicle. The degenerative disease is age-related macular degeneration or Stargardt's macular dystrophy. The present invention further provides a method for preventing photoreceptor degeneration in a vertebrate eye or a method for restoring photoreceptor function in a vertebrate eye comprising administering to the vertebrate an effective amount of a positively charged retinoid compound, e.g., a retinylamine derivative, in a pharmaceutically or ophthamologically acceptable vehicle, slowing chromophore flux in a retinoid cycle in the eye and preventing photoreceptor degeneration in a vertebrate eye or a method for restoring photoreceptor function in the eye.

After absorption of light and photoisomerization of 11-cis-retinal to all-trans-retinal, regeneration of the visual chromophore is a critical step in restoring photoreceptors to their dark-adapted state. This regeneration process, called the retinoid (visual) cycle, takes place in the photoreceptor outer segments and retinal pigmented epithelium (RPE). The present experiments suggested that the regeneration of the chromophore in the eye can occur through a retinyl carbocation intermediate. Evidence is provided to show that isomerization is inhibited by positively charged retinoids. The positively charged retinoids can act as transition state analogs of the isomerization process. Retinylamine (Ret-NH₂) and retinylamine derivatives can potently and selectively inhibit the isomerization step of the retinoid cycle in vitro and in vivo. Ret-NH₂ binds a protein(s) in the RPE microsomes, but it does not bind RPE65, a protein implicated in the isomerization reaction. This new set of inhibitors, positively charged retinoid derivatives, e.g., retinylamine, can regulate chromophore flux more specifically than does 13-cis-retinoic acid (13-cis-RA). The latter has been proposed to treat the symptoms of Stargardt's disease by slowing down the retinoid cycle despite its potential to affect many other tissues than the eye. Importantly, in contrast to 13-cis-RA which can spontaneously isomerize to the all-trans isomer, which in turn activates the nuclear receptors RXR and RAR, Ret-NH₂ does not interact at micromolar concentrations with RXR and RAR. Thus, Ret-NH₂ appears to be a much safer alternative to 13-cis-RA.

11-cis-retinylamine is prepared by reductive amination of 11-cis-retinal. The amine is a strong inhibitor of the isomerase, or isomerohydrolase, protein involved in the visual cycle. In vivo inhibition of isomerase after light bleaching does not lead to the recovery of visual pigment chromophore, thus preventing the formation of retinals and increasing amount of retinyl esters. The retinals are responsible for the accumulation of toxic lipofuscin pigment, A2E, which possess high toxicity towards retinal cells and causes retinal degeneration. This in turn leads to a number of retinal degenerative diseases, for example, Stargardt's disease and age-related macular degeneration, AMD, that leads to vision loss in the patients. Treating the patients with 11-cis-retinylamine can prevent or slow down the formation of A2E and might have the protective properties for the retina.

The present invention provides a method for treating a degenerative disease in a vertebrate eye, comprising administering to the vertebrate an effective amount of a retinylamine derivative in a pharmaceutically or ophthamologically acceptable vehicle. Evidence is presented that the isomerization reaction is potently and reversibly inhibited by a positively charged retinylamine in in vitro assays and in mice. These studies provide supportive evidence that the proposed carbocation mechanism is involved in the isomerization process. Based on these findings, retinylamine analogs appear to be a superior group of compounds compared with 13-cis-RA for the inhibition of the retinoid cycle in vivo. A method for preventing photoreceptor degeneration in a vertebrate eye or a method for restoring photoreceptor function in a vertebrate eye is provided comprising administering to the vertebrate an effective amount of a positively charged retinoid compound, e.g., a retinylamine derivative, in a pharmaceutically or ophthamologically acceptable vehicle, slowing chromophore flux in a retinoid cycle in the eye and preventing photoreceptor degeneration in a vertebrate eye or a method for restoring photoreceptor function in the eye. A method for preventing photoreceptor degeneration in a vertebrate eye or a method for restoring photoreceptor function in a vertebrate eye is provided comprising administering to the vertebrate an effective amount of a retinylamine derivative in a pharmaceutically or ophthamologically acceptable vehicle, slowing chromophore flux in a retinoid cycle in the eye, and preventing photoreceptor degeneration in a vertebrate eye or a method for restoring photoreceptor function in the eye.

“Retinoids” refers to a class of compounds consisting of four isoprenoid units joined in a head to tail manner. See IUPAC-IUB Joint Commission on Biochemical Nomenclature. All retinoids may be formally derived from a monocyclic parent compound containing five carbon-carbon double bonds and a functional group at the terminus of the acyclic portion. The basic retinoid structure is generally subdivided into three segments, namely the polar terminal end (e.g., a terminal amine, alcohol, aldehyde or acid), the conjugated side chain, and the cyclohexenyl ring or the non-polar alkyl side chain. The basic structures of the most common natural retinoids are called retinol, retinaldehyde, and retinoic acid.

“Positively charged retinoid derivative” refers to a retinoid class of compounds, with a positively charged substituent, for example, a primary, secondary, tertiary, or quaternary amine. Further positively charged substituents, include, but are not limited to, amine, disubstituted imidazolium, trisubstituted imidazolium, pyridinium, pyrrolidinium, phosphonium, guanidinium, isouronium, iodonium, or sulfonium (for example SMe₃⁺T).

The synthetic retinoids of the present invention are retinylamine derivatives, for example, 11-cis-retinylamine, 13-cis-retinylamine or 9-cis-retinylamine, or are 11-cis-retinylamine, 13-cis-retinylamine, 9-cis-retinylamine. In certain embodiments, the “synthetic retinoid” is a “synthetic cis retinoid.”

Synthetic retinoids include 11-cis-retinylamine derivatives, 13-cis-retinylamine derivatives, or 9-cis-retinylamine derivatives such as, for example, the following: acyclic retinylamines; retinylamines with modified polyene chain length, such as trienoic or tetraenoic retinylamines; retinylamines with substituted polyene chains, such as alkyl, halogen or heteratom-substituted polyene chains; retinylamines with modified polyene chains, such as trans- or cis-locked polyene chains, or with, for example, allene or alkyne modifications; and retinylamines with ring modifications, such as heterocyclic, heteroaromatic or substituted cycloalkane or cycloalkene rings.

A method for treatment or prophylaxis of a degenerative disease in a vertebrate eye is provided which comprises administering to the vertebrate an effective amount of a positively charged retinoid derivative in a pharmaceutically or ophthamologically acceptable vehicle. In one embodiment, the positively charged retinoid derivative is a retinylamine derivative. In a further aspect, the positively charged retinoid derivative inhibits an isomerization step of the retinoid cycle.

In one embodiment, the method for treatment or prophylaxis of a degenerative disease in a vertebrate eye provides a positively-charged retinoid derivative is a retinoid derivative of formula I:

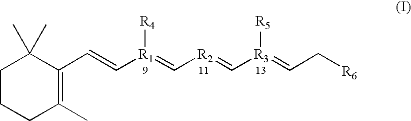

or a stereoisomer, prodrug, pharmaceutically or ophthamologically acceptable salt, hydrate, solvate, acid salt hydrate, N-oxide or isomorphic crystalline form thereof,

wherein at least one or R₁, R₂, R₃, R₄, R₅ or R₆ is a primary, secondary, tertiary or quaternary amine;

wherein R₄ or R₅ are, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₇R₈⁺X⁻, CH₂—NR₇R₈, NR₇R₈, or NR₇R₈R₉⁺X⁻;

wherein R₆ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₇R₈⁺X⁻, OR₇, SR₇, CH₂—NR₇R₉, NR₇R₈, or NR₇R₈R₉⁺X⁻;

wherein R₇, R₈, and R₉ are independently, H, C₁ to C₆ alkyl, C₂ to C₆ alkenyl, C₂ to C₆ alkynyl, or C₃ to C₄ cycloalkyl, OH, or OR₁₀, wherein R₁₀ is C₁ to C₆ alkyl; and X is an anion, Cl, Br, I, SO₃H, or P(O)₂(OH)₂.

In one aspect, the positively-charged retinoid derivative is all trans-isomer, 9-cis-isomer, 11-cis-isomer, 13-cis-isomer, 9,11-di-cis-isomer, 9,13-di-cis-isomer, 11,13-di-cis-isomer, or 9,11,13-tri-cis-isomer.

In a further aspect, the positively-charged retinoid derivative is 11-cis retinylamine. In a further aspect, the positively-charged retinoid derivative is 9-cis retinylamine, 13-cis retinylamine, or all trans retinylamine.

In a further embodiment, the method for treatment or prophylaxis of a degenerative disease in a vertebrate eye provides a positively-charged retinoid derivative is a retinoid derivative of formula II:

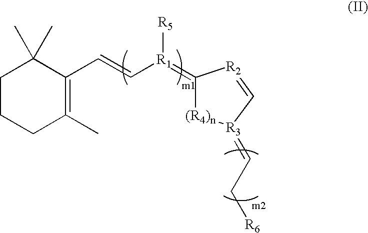

or a stereoisomer, prodrug, pharmaceutically or ophthamologically acceptable salt, hydrate, solvate, acid salt hydrate, N-oxide or isomorphic crystalline form thereof,

wherein n is 1, 2, 3, or 4;

m₁ plus m₂ equals 1, 2, or 3; and

wherein at least one or R₁, R₂, R₃, R₄, R₅ or R₆ is a primary, secondary, tertiary or quaternary amine;

wherein R₅ is, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₇R₈⁺X⁻, CH₂—NR₇R₈, NR₇R₈, or NR₇R₈R₉⁺X⁻;

wherein R₆ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₇R₈⁺X), OR₇, SR₇, CH₂—NR₇R₈, NR₇R₈, or NR₇R₈R₉⁺X⁻;

wherein R₇ R₈, and R₉ are independently, H, C₁ to C₆ alkyl, C₂ to C₆ alkenyl, C₂ to C₆ alkynyl, or C₃ to C₄ cycloalkyl, OH, or OR₁₀, wherein R₁₀ is C₁ to C₆ alkyl; and X is an anion, Cl, Br, I, SO₃H, or P(O)₂(OH)₂.

In a further embodiment, the method for treatment or prophylaxis of a degenerative disease in a vertebrate eye provides a positively-charged retinoid derivative is a retinoid derivative of formula III:

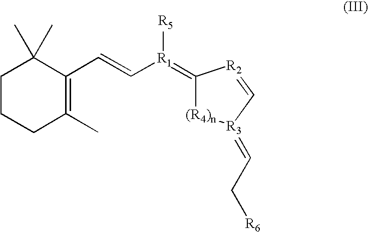

or a stereoisomer, prodrug, pharmaceutically or ophthamologically acceptable salt, hydrate, solvate, acid salt hydrate, N-oxide or isomorphic crystalline form thereof,

wherein n is 1, 2, 3, or 4; and

wherein at least one of R₁, R₂, R₃, R₄, R₅ or R₆ is a primary, secondary, tertiary or quaternary amine;

wherein R₅ is, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₇R₈⁺X⁻, CH₂—NR₇R₈, NR₇R₈, or NR₇R₈R₉⁺X⁻;

wherein R₆ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₇R₈⁺X⁻, OR₇, SR₇, CH₂—NR₇R₉, NR₇R₈, or NR₇R₈R₉⁺X⁻;

wherein R₇ R₈, and R₉ are independently, H, C₁ to C₆ alkyl, C₂ to C₆ alkenyl, C₂ to C₆ alkynyl, or C₃ to C₄ cycloalkyl, OH, or OR₁₀, wherein R₁₀ is C₁ to C₆ alkyl; and X is an anion, Cl, Br, I, SO₃H, or P(O)₂(OH)₂.

In one aspect, the positively-charged retinoid derivative is 11-cis locked retinylamine.

In a further embodiment, the method for treatment or prophylaxis of a degenerative disease in a vertebrate eye provides a positively-charged retinoid derivative is a retinoid derivative of formula IV:

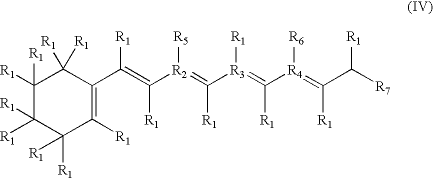

or a stereoisomer, prodrug, pharmaceutically or ophthamologically acceptable salt, hydrate, solvate, acid salt hydrate, N-oxide or isomorphic crystalline form thereof,

R₁ is, independently, hydrogen, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, OR₈, SR₈, or NR₈R₉, wherein R₈ and R₉ are, independently, H, C₁ to C₆ alkyl;

wherein at least one of R₂, R₃, R₄, R₅, R₆ or R₇ is a primary, secondary, tertiary or quaternary amine;

wherein R₅ or R₆ are, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₁₀R₁₁⁺X⁻, CH₂—NR₁₀R₁₁, NR₁₀R₁₁, or NR₁₀R₁₁R₁₂⁺X⁻;

wherein R₇ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₁₀R₁₁⁺X⁻, OR₁₀, SR₁₀, CH₂—NR₁₀R₁₁, NR₁₀R₁₁, or NR₁₀R₁₁, R₁₂⁺X⁻;

wherein R₁₀, R₁₁, and R₁₂ are independently, H, C₁ to C₆ alkyl, C₂ to C₆ alkenyl, C₂ to C₆ alkynyl, or C₃ to C₄ cycloalkyl, OH, or OR₁₃, wherein R₁₃ is C₁ to C₆ alkyl; and X is an anion, Cl, Br, I, SO₃H, or P(O)₂(OH)₂.

In one aspect, the positively-charged retinoid derivative is all trans-isomer, 9-cis-isomer, 11-cis-isomer, 13-cis-isomer, 9,11-di-cis-isomer, 9,13-di-cis-isomer, and 11,13-di-cis-isomer, or 9,11,13-tri-cis-isomer.

In a further embodiment, the method for treatment or prophylaxis of a degenerative disease in a vertebrate eye provides a positively-charged retinoid derivative is a retinoid derivative of formula V:

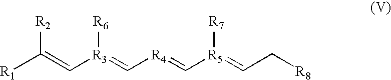

or a stereoisomer, prodrug, pharmaceutically or ophthamologically acceptable salt, hydrate, solvate, acid salt hydrate, N-oxide or isomorphic crystalline form thereof,

R₁ and R₂ are, independently, lower alkyl, straight chain alkyl, linear, iso-alkyl, sec-alkyl, tert-alkyl, C₁ to C₆ branched chain alkyl, substituted alkyl groups, substituted branched chain alkyl, hydroxyl, hydroalkyl, amine, or amide;

wherein at least one of R₃, R₄, R₅, R₆, R₇, or R₉ is a primary, secondary, tertiary or quaternary amine;

wherein R₆ or R₇ are, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₉R₁₀⁺X⁻, CH₂—NR₉R₁₀, NR₉R₁₀, or NR₉R₁₀R₁₁⁺X⁻;

wherein R₉ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₉R₁₀⁺X⁻, OR₇, SR₇, CH₂—NR₉R₁₀, NR₉R₁₀, or NR₉R₁₀R₁₁⁺X⁻;

wherein R₉ R₁₀, and R₁₁ are independently, H, C₁ to C₆ alkyl, C₂ to C₆ alkenyl, C₂ to C₆ alkynyl, or C₃ to C₄ cycloalkyl, OH, or OR₁₂, wherein R₁₂ is C₁ to C₆ alkyl; and X is an anion, Cl, Br, I, SO₃H, or P(O)₂(OH)₂.

In one aspect, the degenerative disease is a result of lipofuscin pigment accumulation in the eye. In a further aspect, the degenerative disease is a result of N-retinylidene-N-retinylethanolamine accumulation in the eye. In a detailed aspect, the degenerative disease is age-related macular degeneration or Stargardt's macular dystrophy.

In a further aspect the retinoid derivative is locally administered to the eye, and further wherein the retinoid derivative is locally administered by eye drops, intraocular injection or periocular injection. The retinoid derivative can also be orally administered to the vertebrate.

A method for preventing photoreceptor degeneration in a vertebrate eye or a method for restoring photoreceptor function in a vertebrate eye is provided which comprises administering to the vertebrate an effective amount of a positively charged retinoid compound in a pharmaceutically or ophthamologically acceptable vehicle, slowing chromophore flux in a retinoid cycle in the eye, and preventing photoreceptor degeneration in a vertebrate eye or a method for restoring photoreceptor function in the eye. In one embodiment, the positively charged retinoid derivative is a retinylamine derivative. In a further aspect, the positively charged retinoid derivative inhibits an isomerization step of the retinoid cycle.

In one embodiment, the method for preventing photoreceptor degeneration in a vertebrate eye or a method for restoring photoreceptor function in a vertebrate eye provides positively-charged retinoid compound is a retinoid derivative of formula I:

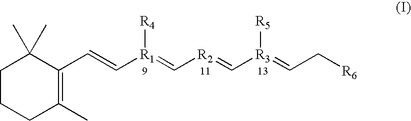

or a stereoisomer, prodrug, pharmaceutically or ophthamologically acceptable salt, hydrate, solvate, acid salt hydrate, N-oxide or isomorphic crystalline form thereof,

wherein at least one of R₁, R₂, R₃, R₄, R₅ or R₆ is a primary, secondary, tertiary or quaternary amine;

wherein R₄ or R₅ are, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₇R₈⁺X⁻, CH₂—NR₇R₈, NR₇R₈, or NR₇R₈R₉⁺X⁻;

wherein R₆ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₇R₈⁺X⁻, OR₇, SR₇, CH₂—NR₇R₈, NR₇R₈, or NR₇R₈R₉⁺X⁻;

wherein R₇ R₈, and R₉ are independently, H, C₁ to C₆ alkyl, C₂ to C₆ alkenyl, C₂ to C₆ alkynyl, or C₃ to C₄ cycloalkyl, OH, or OR₁₀, wherein R₁₀ is C₁ to C₆ alkyl; and X is an anion, Cl, Br, I, SO₃H, or P(O)₂(OH)₂.

In one aspect, the positively-charged retinoid compound is 11-cis retinylamine. In a further aspect, the positively-charged retinoid compound is all trans-isomer, 9-cis-isomer, 11-cis-isomer, 13-cis-isomer, 9,11-di-cis-isomer, 9,13-di-cis-isomer, 11,13-di-cis-isomer, or 9,11,13-tri-cis-isomer.

In a further embodiment, the method for preventing photoreceptor degeneration in a vertebrate eye or a method for restoring photoreceptor function in a vertebrate eye provides a positively-charged retinoid compound is a retinoid derivative of formula I:

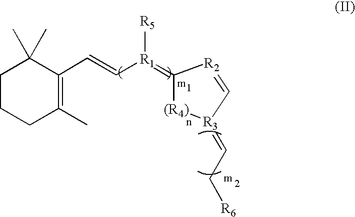

or a stereoisomer, prodrug, pharmaceutically or ophthamologically acceptable salt, hydrate, solvate, acid salt hydrate, N-oxide or isomorphic crystalline form thereof,

wherein n is 1, 2, 3, or 4;

m₁ plus m₂ equals 1, 2, or 3; and

wherein at least one of R₁, R₂, R₃, R₄, R₅ or R₆ is a primary, secondary, tertiary or quaternary amine;

wherein R₅ is, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₇R₈⁺X⁻, CH₂—NR₇R₈, NR₇R₈, or NR₇R₈R₉⁺X⁻;

wherein R₆ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₇R₈⁺X⁻, OR₇, SR₇, CH₂—NR₇R₈, NR₇R₈, or NR₇R₈R₉⁺X);

wherein R₇, R₈, and R₉ are independently, H, C₁ to C₆ alkyl, C₂ to C₆ alkenyl, C₂ to C₆ alkynyl, or C₃ to C₄ cycloalkyl, OH, or OR₁₀, wherein R₁₀ is C₁ to C₆ alkyl; and X is an anion, Cl, Br, I, SO₃H, or P(O)₂(OH)₂.

In a manner embodiment, the method for preventing photoreceptor degeneration in a vertebrate eye or a method for restoring photoreceptor function in a vertebrate eye provides a positively-charged retinoid compound is a retinoid derivative of formula III:

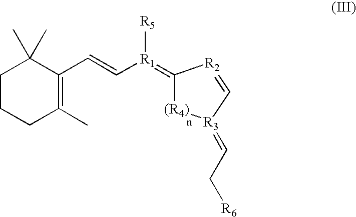

or a stereoisomer, prodrug, pharmaceutically or ophthamologically acceptable salt, hydrate, solvate, acid salt hydrate, N-oxide or isomorphic crystalline form thereof,

wherein n is 1, 2, 3, or 4; and

wherein at least one of R₁, R₂, R₃, R₄, R₅ or R₆ is a primary, secondary, tertiary or quaternary amine;

wherein R₅ is, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₇R₈⁺X⁻, CH₂—NR₇R₈, NR₇R₈, or NR₇R₈R₉⁺X⁻;

wherein R₆ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₇R₈⁺X⁻, OR₇, SR₇, CH₂—NR₇R₈, NR₇R₈, or NR₇R₈R₉⁺X⁻;

wherein R₇ R₈, and R₉ are independently, H, C₁ to C₆ alkyl, C₂ to C₆ alkenyl, C₂ to C₆ alkynyl, or C₃ to C₄ cycloalkyl, OH, or OR₁₀, wherein R₁₀ is C₁ to C₆ alkyl; and X is an anion, Cl, Br, I, SO₃H, or P(O)₂(OH)₂.

In a further aspect, the positively-charged retinoid compound is 11-cis locked retinylamine.

In a further embodiment, the method for preventing photoreceptor degeneration in a vertebrate eye or a method for restoring photoreceptor function in a vertebrate eye provides a positively-charged retinoid compound is a retinoid derivative of formula IV:

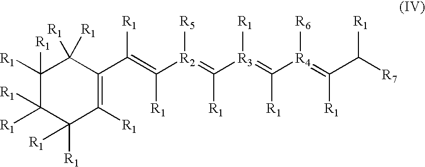

or a stereoisomer, prodrug, pharmaceutically or ophthamologically acceptable salt, hydrate, solvate, acid salt hydrate, N-oxide or isomorphic crystalline form thereof,

R₁ is, independently, hydrogen, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, OR₈, SR₈, or NR₈R₉, wherein R₈ and R₉ are, independently, H, C₁ to C₆ alkyl;

wherein at least one of R₂, R₃, R₄, R₅, R₆ or R₇ is a primary, secondary, tertiary or quaternary amine;

wherein R₅ or R₆ are, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₁₀R₁₁⁺X⁻, CH₂—NR₁₀R₁₁, NR₁₀R₁₁, or NR₁₀R₁₁R₁₂⁺X⁻;

wherein R₇ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₁₀R₁₁⁺X⁻, OR₁₀, SR₁₀, CH₂—NR₁₀R₁₁, NR₁₀R₁₁, or NR₁₀R₁₁R₁₂⁺X⁻;

wherein R₁₀, R₁₁, and R₁₂ are independently, H, C₁ to C₆ alkyl, C₂ to C₆ alkenyl, C₂ to C₆ alkynyl, or C₃ to C₄ cycloalkyl, OH, or OR₁₃, wherein R₁₃ is C₁ to C₆ alkyl; and X is an anion, Cl, Br, I, SO₃H, or P(O)₂(OH)₂

In a further aspect, the retinoid derivative is all trans-isomer, 9-cis-isomer, 11-cis-isomer, 13-cis-isomer, 9,11-di-cis-isomer, 9,13-di-cis-isomer, 11,13-di-cis-isomer, or 9,11,13-tri-cis-isomer.

In a further embodiment, the method for preventing photoreceptor degeneration in a vertebrate eye or a method for restoring photoreceptor function in a vertebrate eye provides a positively-charged retinoid compound comprises a retinoid derivative of formula V:

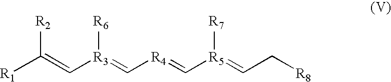

or a stereoisomer, prodrug, pharmaceutically or ophthamologically acceptable salt, hydrate, solvate, acid salt hydrate, N-oxide or isomorphic crystalline form thereof,

R₁ and R₂ are, independently, lower alkyl, straight chain alkyl, linear, iso-alkyl, sec-alkyl, tert-alkyl, C₁ to C₆ branched chain alkyl, substituted alkyl groups, substituted branched chain alkyl, hydroxyl, hydroalkyl, amine, or amide;

wherein at least one of R₃, R₄, R₅, R₆, R₇, or R₈ is a primary, secondary, tertiary or quaternary amine;

wherein R₆ or R₇ are, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₉R₁₀⁺X⁻, CH₂—NR₉R₁₀, NR₉R₁₀, or NR₉R₁₀OR₁₁⁺X⁻;

wherein R₈ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₉R₁₀⁺X⁻, OR₇, SR₇, CH₂—NR₉R₁₀, NR₉R₁₀, or NR₉R₁₀R₁₁⁺X⁻;

wherein R₉ R₁₀, and R₁₁ are independently, H, C₁ to C₆ alkyl, C₂ to C₆ alkenyl, C₂ to C₆ alkynyl, or C₃ to C₄ cycloalkyl, OH, or OR₁₂, wherein R₁₂ is C₁ to C₆ alkyl; and X is an anion, Cl, Br, I, SO₃H, or P(O)₂(OH)₂

In one aspect, the degenerative disease is a result of lipofuscin pigment accumulation in the eye. In a further aspect, the degenerative disease is a result of N-retinylidene-N-retinylethanolamine accumulation in the eye. In a detailed aspect, the degenerative disease is age-related macular degeneration or Stargardt's macular dystrophy.

In a further aspect the retinoid derivative is locally administered to the eye, and further wherein the retinoid derivative is locally administered by eye drops, intraocular injection or periocular injection. The retinoid derivative can also be orally administered to the vertebrate.

In a specific embodiment the synthetic retinoid is 10-ethyl-3,7-dimethyl-dodeca-2,4,6,8-tetraenylamine.

Methods of making synthetic retinoid compounds and derivatives are disclosed in, for example, the following references: Anal. Biochem. 272:232-42, 1999; Angew, Chem. 36:2089-93, 1997; Biochemistry 14:3933-41, 1975; Biochemistry 21:384-93, 1982; Biochemistry 28:2732-39, 1989; Biochemistry 33:408-16, 1994; Biochiemistry 35:6257-62, 1996; Bioorganic Chemistry 27:372-82, 1999; Biophys. Chem. 56:31-39, 1995; Biophys. J. 56:1259-65, 1989; Biophys. J. 83:3460-6, 2002; Chemistry 7:4198-204, 2001; Chemistry (Europe) 5:1172-75, 1999; FEBS 158:1, 1983; J. American Chem. Soc. 104:3214-16, 1982; J. Am. Chem. Soc. 108:6077-78, 1980, J. Am. Chem. Soc. 109:0163, 1987; J. Am. Chem. Soc. 112:7779-82, 1990; J. Am. Chem. Soc. 119:5758-59, 1997; J. Am. Chem. Soc. 121:5803-04, 1999; J. American Chem. Soc. 123:10024-29, 2001; J. American Chem. Soc. 124:7294-302, 2002; J. Biol. Chem. 276:26148-53, 2001; J. Biol. Chem. 277:42315-24, 2004; J. Chem. Soc.—Perkin T. 1:1773-77, 1997; J. Chem. Soc.—Perkin T. 1:2430-39, 2001; J. Org. Chem. 49:649-52, 1984; J. Org. Chem. 58:3533-37, 1993; J. Physical Chemistry B 102:2787-806, 1998; Lipids 8:558-65; Photochem. Photobiol. 13:259-83, 1986; Photochem. Photobiol. 44:803-07, 1986; Photochem. Photobiol. 54:969-76, 1991; Photochem. Photobiol. 60:64-68 (1994); Photochem. Photobiol. 65:1047-55, 1991; Photochem. Photobiol. 70:111-15, 2002; Photochem. Photobiol. 76:606-615, 2002; Proc. Natl. Acad. Sci. USA 88:9412-16, 1991; Proc. Natl. Acad. Sci. USA 90:4072-76, 1993; Proc. Natl. Acad. Sci. USA 94:13442-47, 1997; and Proc. R. Soc. Lond. Series B, Biol. Sci. 233(1270):55-76, 1988 (the disclosures of which are incorporated by reference herein).

Retinyl esters can be formed by methods known in the art such as, for example, by acid-catalyzed esterification of a retinol with a carboxylic acid, by reaction of retinal with carboxylic acid in the presence of coupling reagents such as dicyclohexylcarbodiimide, as similar, or by Mitsunobu reaction between retinol and carboxylic acid in the presence of triphenylphosphine and diethyl(isopropyl)azodicarboxylate, by reaction of an acyl halide with a retinol, by base-catalyzed reaction of acid anhydride with retinol, by transesterification of a retinyl ester with a carboxylic acid, by reaction of a primary halide with a carboxylate salt of a retinoic acid, or the like. In an exemplary embodiment, retinyl esters can be formed by acid-catalyzed esterification of a retinol with a carboxylic acid, such as, acetic acid, propionic acid, butyric acid, valeric acid, caproic acid, caprylic acid, pelargonic acid, capric acid, lauric acid, oleic acid, stearatic acid, palmitic acid, myristic acid, linoleic acid, succinic acid, fumaric acid or the like. In another exemplary embodiment, retinyl esters can be formed by reaction of an acyl halide with a retinol (see, e.g., Van Hooser et al., Proc. Natl. Acad. Sci. USA, 97:8623-28, 2000). Suitable acyl halides include, for example, acetyl chloride, palmitoyl chloride, or the like.

Retinyl ethers can be formed by methods known in the art, such as for example, reaction of a retinol with a primary alkyl halide.

In certain embodiments, trans-retinoids can be isomerized to cis-retinoids by exposure to UV light. For example, all-trans-retinal, all-trans-retinol, all-trans-retinyl ester or all-trans-retinoic acid can be isomerized to 9-cis-retinal, 9-cis-retinol, 9-cis-retinyl ester or 9-cis-retinoic acid, respectively. trans-Retinoids can be isomerized to 9-cis-retinoids by, for example, exposure to a UV light having a wavelength of about 365 nm, and substantially free of shorter wavelengths that cause degradation of cis-retinoids, as further described herein.

Retinyl acetans and hemiacetals can be prepared, for example, by treatment of 9-cis- and 11-cis-retonals with alcohols in the presence of acid catalysts. Water formed during reaction is removed, for example by Al₂O₃ of a molecular sieve.

Retinyl oximes can be prepared, for example, by reaction of a retinal with hydroxylamine, O-methyl- or O-ethylhydroxyl amine, or the like.

A synthetic retinylamine derivative can be administered to vertebrate eyes having a retinoid excess (e.g., an excess of 11-cis-retinol or 11-cis-retinal), an excess of retinoid waste products or intermediates in the recycling of all-trans-retinal, or the like. The vertebrate eye typically comprises a wild-type opsin protein. Methods of determining endogenous retinoid levels in a vertebrate eye, and an excess or deficiency of such retinoids, are disclosed in, for example, U.S. Provisional Patent Application No. 60/538,051 (filed Feb. 12, 2004) (the disclosure of which is incorporated by reference herein). Other methods of determining endogenous retinoid levels in a vertebrate eye, and an excess of such retinoids, include for example, analysis by high pressure liquid chromatography (HPLC) of retinoids in a sample from a subject. For example, retinoid levels or an excess in such levels can be determined from a blood sample from a subject.

In an exemplary embodiment, a blood sample can be obtained from a subject and retinoid types and levels in the sample can be separated and analyzed by normal phase high pressure liquid chromatography (HPLC) (e.g., with a HP1100 HPLC and a Beckman, Ultrasphere-Si, 4.6 mm×250 mm column using 10% ethyl acetate/90% hexane at a flow rate of 1.4 ml/minute). The retinoids can be detected by, for example, detection at 325 nm using a diode-array detector and HP Chemstation A.03.03 software. An excess in retinoids can be determined, for example, by comparison of the profile of retinoids in the sample with a sample from a normal subject.

As used herein, increased or excessive levels of endogenous retinoid, such as 11-cis-retinol or 11-cis-retinal, refer to levels of endogenous retinoid lower than those found in a healthy eye of a vertebrate of the same species. A synthetic retinylamine derivative can spare the requirement for endogenous retinoid.

As used herein, “prophylactic” and “prophylactically” refer to the administration of a synthetic retinylamine derivative to prevent degeneration or further degeneration or deterioration or further deterioration of the vertebrate visual system, as compared with a comparable vertebrate visual system not receiving the synthetic retinylamine derivative. The term “restore” refers to a long-term (e.g., as measured in weeks or months) improvement in photoreceptor function in a vertebrate visual system, as compared with a comparable vertebrate visual system not receiving the synthetic retinylamine derivative. The term “stabilize” refers to minimization of additional degeneration or additional degradation in a vertebrate visual system, as compared with a comparable vertebrate visual system not receiving the synthetic retinylamine derivative.

In one aspect, the vertebrate eye is characterized as having Leber Congenital Amaurosis (“LCA”). This disease is a very rare childhood condition that effects children from birth or shortly there after. It affects both rods and cones in the eye. For example, certain mutations in the genes encoding RP65 and LRAT proteins are involved in LCA. Mutations in both genes result in a person's inability to make 11-cis-retinal in adequate quantities. Thus, 11-cis-retinal is either absent or present in reduced quantities. In RP65-defective individuals, retinyl esters build up in the RPE. LRAT-defective individuals are unable to make esters and subsequently secrete any excess retinoids. For LCA, a synthetic retinal derivative can be used to replace the absent or depleted 11-cis-retinal.

In another aspect, the vertebrate eye is characterized as having Stargardt's disease or Stargardt's macular degeneration. In Stargardt's disease, associated with mutations in the ABCR transporter, the accumulation of all-trans-retinal has been proposed to be responsible for the formation, of a lipofuscin pigment, A2E, which is toxic towards retinal cells and causes retinal degeneration and consequently loss of vision.

In yet another aspect, the vertebrate eye is characterized as having age-related macular degeneration (“AMD”). In various embodiments, AMD can be wet or dry forms. In AMD, vision loss occurs when complications late in the disease either cause new blood vessels to grow under the retina or the retina atrophies. Without intending to be bound by any particular theory, the accumulation of all-trans-retinal has been proposed to be responsible for the formation of a lipofuscin pigment, N-retinylidene-N-retinylethanolamine (A2E), which is toxic towards retinal cells and causes retinal degeneration and consequently loss of vision.

In the vertebrate eye, for example, a mammalian eye, the formation of A2E is a light-dependent process and its accumulation leads to a number of negative effects in the eye. These include destabilization of retinal pigment epithelium (RPE) membranes, sensitization of cells to blue-light damage, and impaired degradation of phospholipids. Products of A2E oxidation by molecular oxygen (oxiranes) were even shown to induce DNA damage in cultured RPE cells. All these factors lead to a gradual decrease in visual acuity and eventually to vision loss. If it were possible to reduce the formation of retinals during vision processes, it would lead to decreased amounts of A2E in the eye. This would delay the aging of the RPE and retina and would slow down or prevent vision loss. Treating patients with 11-cis-retinylamine can prevent or slow down the formation of A2E and can have protective properties for the retina.

“Treating” or “treatment” refers to any indicia of success in the treatment or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination. Accordingly, the term “treating” includes the administration of the compounds or agents of the present invention to treat pain, hyperalgesia, allodynia, or nociceptive events. Accordingly, the term “treating” includes the administration of the compounds or agents of the present invention to prevent or delay, to alleviate, or to arrest or inhibit development of the symptoms or conditions associated with pain, hyperalgesia, allodynia, nociceptive events, or other disorders. The term “therapeutic effect” refers to the reduction, elimination, or prevention of the disease, symptoms of the disease, or side effects of the disease in the subject.

“Vertebrate”, “subject”, or “patient” or “mammal” refer to any vertebrate or mammalian patient or subject to which the compositions of the invention can be administered. The term “vertebrate” or “mammal” refers to human patients and non-human primates, as well as experimental animals such as rabbits, rats, and mice, and other animals. In an exemplary embodiment, of the present invention, to identify subject patients for treatment according to the methods of the invention, accepted screening methods are employed to determine risk factors associated with a targeted or suspected disease or condition or to determine the status of an existing disease or condition in a subject, e.g., Stargardt's macular degeneration or age-related macular degeneration. These screening methods include, for example, conventional work-ups to determine risk factors that can be associated with the targeted or suspected disease or condition. These and other routine methods allow the clinician to select patients in need of therapy using the methods and formulations of the invention.

The compounds employed in the methods of the present invention may exist in prodrug form. “Prodrug” is intended to include any covalently bonded carriers which release the active parent drug, for example, as according to Formula I, II, IV, I, or V, or other formulas or compounds employed in the methods of the present invention in vivo when such prodrug is administered to a mammalian subject. Since prodrugs are known to enhance numerous desirable qualities of pharmaceuticals (e.g., solubility, bioavailability, manufacturing, etc.) the compounds employed in the present methods may, if desired, be delivered in prodrug form. Thus, the present invention contemplates methods of delivering prodrugs. Prodrugs of the compounds employed in the present invention, for example Formula I, H, III, IV, or V, may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, or phenethyl esters.

Examples of prodrugs of retinylamines further include, but are not limited to, an amide derivative, thioamide derivative, carbamate derivative, thiocarbamate derivative, imide derivative, sulphonamide derivative, imine derivative, protonated imine derivative, isocyanate derivative, or isothiocyanate derivative of retinylamine. See for example, FIG. 11. The prodrug can be, for example, retinylamide, retinylthioamide, retinylcarbamate, or retinylthiocarbamate.

The compounds are preferably combined with a pharmaceutical carrier selected on the basis of the chosen route of administration and standard pharmaceutical practice as described, for example, in Remington's Pharmaceutical Sciences (Mack Pub. Co., Easton, Pa., 1980), the disclosure of which is hereby incorporated herein by reference, in its entirety.

Although the compounds of the present invention may be administered as the pure chemicals, it is preferable to present the active ingredient as a pharmaceutical composition. The invention thus further provides a pharmaceutical composition comprising one or more retinylamine compounds, or a stereoisomer, prodrug, pharmaceutically or ophthamologically acceptable salt, hydrate, solvate, acid salt hydrate, N-oxide or isomorphic crystalline form thereof, of the present invention, together with one or more pharmaceutically acceptable carriers therefore and, optionally, other therapeutic and/or prophylactic ingredients. The carrier(s) must be acceptable in the sense of being compatible with the other ingredients of the composition and not deleterious to the recipient thereof.

Stargardt's macular degeneration, a recessive inherited disease, is an inherited blinding disease of children. The primary pathologic defect in Stargardt's disease is also an accumulation of toxic lipofuscin pigments such A2E in cells of the retinal pigment epithelium (KPE). This accumulation appears to be responsible for the photoreceptor death and severe visual loss found in Stargardt's patients. Retinylamine can slow the synthesis of 11-cis-retinaldehyde (11cRAL) and regeneration of -5-rhodopsin by inhibiting isomerase in the visual cycle. Light activation of rhodopsin results in its release of all-trans-retinal, which constitutes the first reactant in A2E biosynthesis.

Retinylamine derivatives can also block age-dependent accumulation of lipofuscin in wild-type mice. Further, the treatment with retinylamine can inhibit lipofuscin accumulation and thus delay the onset of visual loss in Stargardt's and AMD patients. The retinylamine derivative is expected to have a low toxicity. Finally, retinylamine can be an effective treatment for other forms of retinal or macular degeneration associated with lipofuscin accumulation.

Research has been done on the prevention of A2E accumulation in the RPE. This includes treatment with 13-cis-retinoic acid (Accutane® or Isotretinoin), a drug commonly used for the treatment of acne and an inhibitor of 11-cis-retinol dehydrogenase. The major drawback in this proposed treatment is that 13-cis-retinoic acid can easily isomerize to all-trans-retinoic acid. All-trans-retinoic acid is a very potent teratogenic compound that causes adverse effects cell proliferation and development. Retinoic acid also accumulates in the liver and may be a contributing factor in liver diseases.

Administration of a synthetic retinylamine derivative to the vertebrate eye can prevent formation of the lipofuscin pigment, A2E, which is toxic towards retinal cells and causes retinal degeneration. In certain embodiments, administration of a synthetic retinylamine derivative can lessen the production of waste products, e.g., lipofuscin pigment, A2E, and reduce or slow vision loss (e.g., choroidal neovascularization and/or chorioretinal atrophy).

In yet other aspects, a synthetic retinylamine derivative is administered to a subject such as a human with a mutation in the ABCR transporter in the eye. The synthetic retinylamine derivative can also be administered to an aging subject, such as a human. As used herein, an aging human subject is typically at least 45, or at least 50, or at least 60, or at least 65 years old. In Stargardt's disease, associated with mutations in the ABCR transporter, the accumulation of all-trans-retinal has been proposed to be responsible for the formation of a lipofuscin pigment, A2E, which is toxic towards retinal cells and causes retinal degeneration and consequently loss of vision. Without wishing to be bound by theory, retinyl amine derivatives can be a strong inhibitor of the isomerohydrolase protein involved in the visual cycle. Treating patients with a retinylamine derivative, e.g., 11-cis-retinylamine can prevent or slow down the formation of A2E and can have protective properties for normal vision.

Synthetic retinylamine derivatives can be administered to human or other non-human vertebrates. In certain embodiments, the synthetic retinylamine derivative is substantially pure, in that is contains less than about 5% or less than about 1%, or less than about 0.1%, other retinoids. In other embodiments, a combination of synthetic retinylamine derivatives can be administered.

Synthetic retinylamine derivatives can be delivered to the eye by any suitable means, including, for example, oral or local administration. Modes of local administration can include, for example, eye drops, intraocular injection or periocular injection. Periocular injection typically involves injection of the synthetic retinylamine derivative into the conjunctiva or to the tennon (the fibrous tissue overlying the eye). Intraocular injection typically involves injection of the synthetic retinylamine derivative into the vitreous. In certain embodiments, the administration is non-invasive, such as by eye drops or oral dosage form.

Synthetic retinylamine derivatives can be formulated for administration using pharmaceutically acceptable vehicles as well as techniques routinely used in the art. A vehicle is selected according to the solubility of the synthetic retinylamine derivative. Suitable opthalmological compositions include those that are administrable locally to the eye, such as by eye drops, injection or the like. In the case of eye drops, the formulation can also optionally include, for example, opthalmologically compatible agents such as isotonizing agents such as sodium chloride, concentrated glycerin, and the like; buffering agents such as sodium phosphate, sodium acetate, and the like; surfactants such as polyoxyethylene sorbitan mono-oleate (also referred to as Polysorbate 80), polyoxyl stearate 40, polyoxyethylene hydrogenated castor oil, and the like; stabilization agents such as sodium citrate, sodium edentate, and the like; preservatives such as benzalkonium chloride, parabens, and the like; and other ingredients. Preservatives can be employed, for example, at a level of from about 0.001 to about 1.0% weight/volume. The pH of the formulation is usually within the range acceptable to ophthalmologic formulations, such as within the range of about pH 4 to 8.

For injection, the synthetic retinylamine derivative can be provided in an injection grade saline solution, in the form of an injectable liposome solution, or the like. Intraocular and periocular injections are known to those skilled in the art and are described in numerous publications including, for example, Spaeth, Ed., Ophthalmic Surgery: Principles of Practice, W. B. Sanders Co., Philadelphia, Pa., 85-87, 1990.

Suitable oral dosage forms include, for example, tablets, pills, sachets, or capsules of hard or soft gelatin, methylcellulose or of another suitable material easily dissolved in the digestive tract. Suitable nontoxic solid carriers can be used which include, for example, pharmaceutical grades of mannitol, lactose, starch, magnesium stearate, sodium saccharin, talcum, cellulose, glucose, sucrose, magnesium carbonate, and the like. (See, e.g., Gennaro, Ed., Remington “Pharmaceutical Sciences”, 17 Ed., Mack Publishing Co., Easton, Pa., 1985.

The doses of the synthetic retinylamine derivatives can be suitably selected depending on the clinical status, condition and age of the subject, dosage form and the like. In the case of eye drops, a synthetic retinylamine derivative can be administered, for example, from about 0.01 mg, about 0.1 mg, or about 1 mg, to about 25 mg, to about 50 mg, to about 90 mg per single dose. Eye drops can be administered one or more times per day, as needed. In the case of injections, suitable doses can be, for example, about 0.0001 mg, about 0.001 mg, about 0.01 mg, or about 0.1 mg to about 10 mg, to about 25 mg, to about 50 mg, or to about 90 mg of the synthetic retinylamine derivative, one to four times per week. In other embodiments, about 1.0 to about 30 mg of synthetic retinylamine derivative can be administered one to three times per week.

Oral doses can typically range from about 1.0 to about 1000 mg, one to four times, or more, per day. An exemplary dosing range for oral administration is from about 10 to about 250 mg one to three times per day.

Other embodiments and uses will be apparent to one skilled in the art in light of the present disclosures. The following examples are provided merely as illustrative of various aspects of the invention and shall not be construed to limit the invention in any way.

### EXEMPLARY EMBODIMENTS

### Example 1

### Retinylamine is a Potent and Specific Inhibitor of the Isomerization Reaction

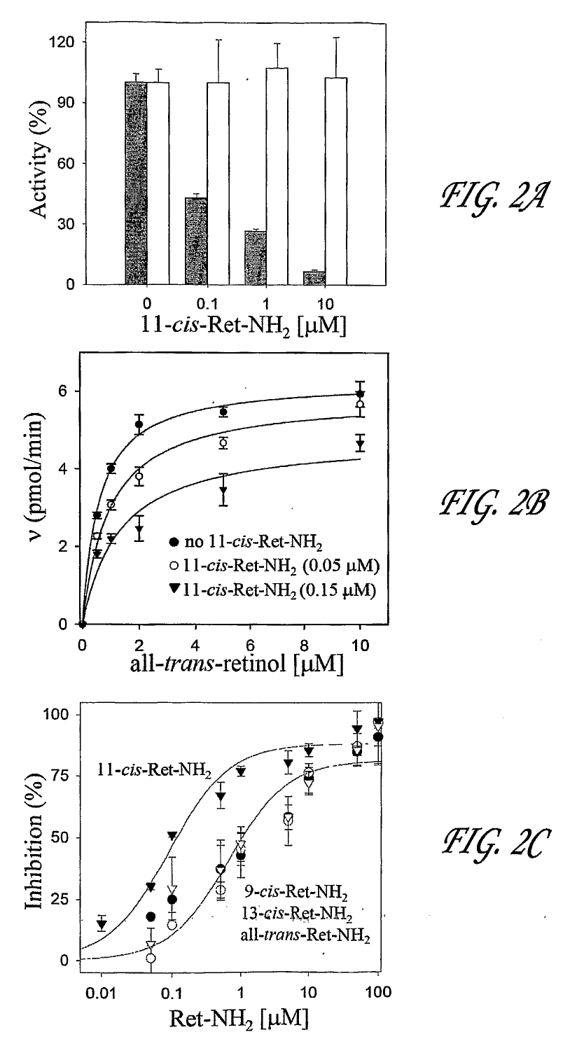

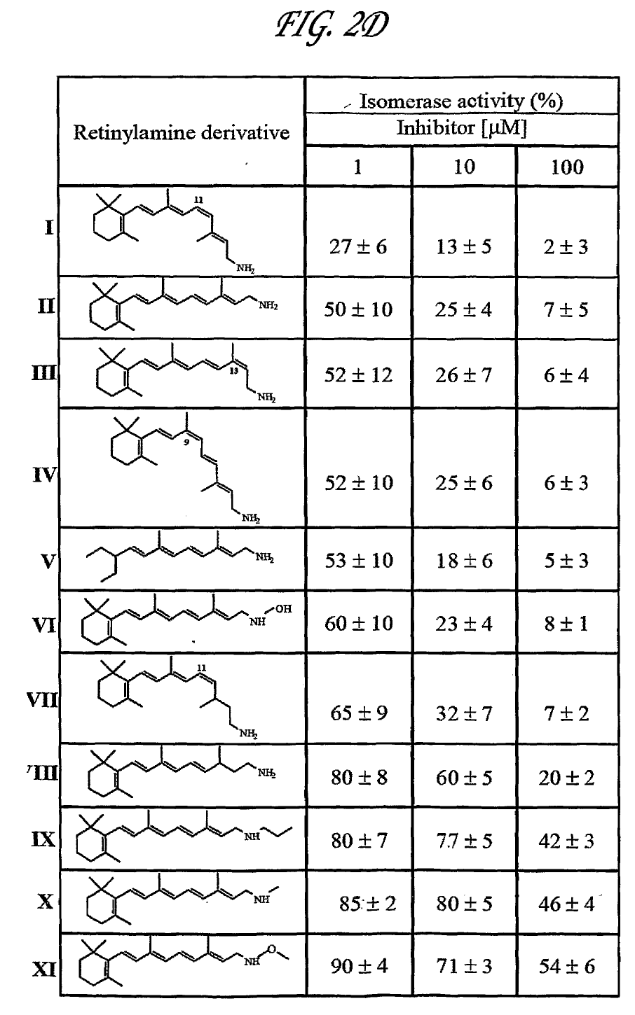

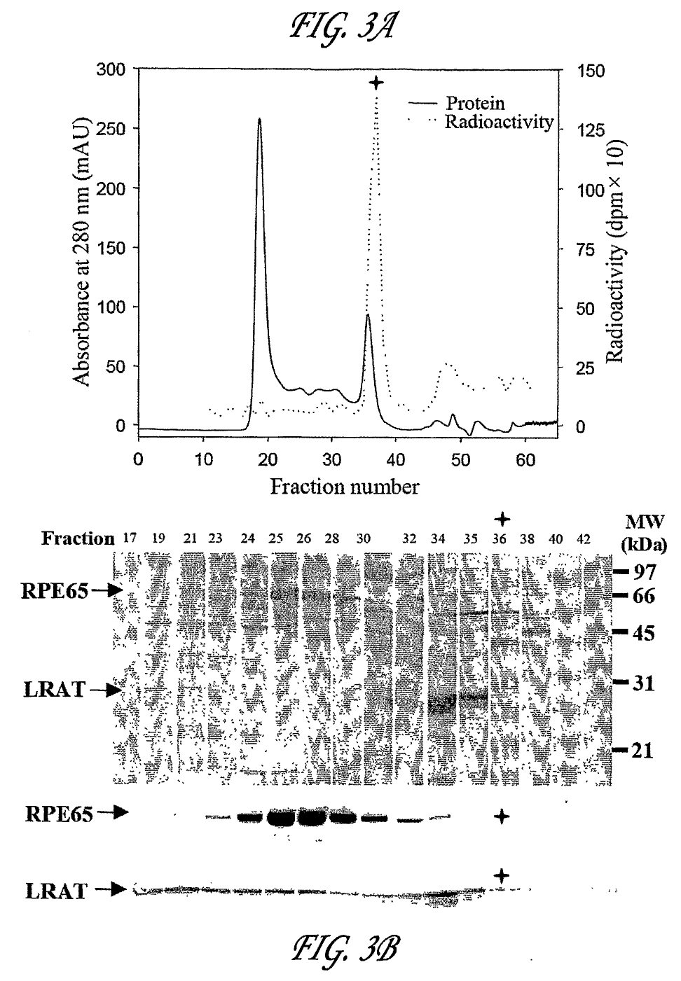

To test if 11-cis-retinylamine (11-cis Ret-NH₂) affects isomerization and esterification, an in vitro assay was employed. In this assay both of the all-trans-retinyl esters formed in the reaction catalyzed by LRAT and 11-cis-retinol production by isomerase were measured (6). Purified RPE microsomes were the source of the enzymes, and all-trans-retinol was the substrate. 11-cis-Ret-NH₂ potently inhibited 11-cis-retinol production with IC₅₀=70 nM (FIG. 2A), whereas the formation of esters was not affected (FIG. 2A). Inhibition of isomerization by Ret-NH₂ is not due to binding of CRALBP or competition with 11-cis-retinol for binding to CRALBP. This is demonstrated by the fact that an increase in the CRALBP concentration from 6 to 30 μM had no effect on the production of 11-cis-retinol. Analysis of retinoids bound to CRALBP isolated from the reaction mixture in the presence of the inhibitor did not reveal any significant levels of 11-cis-Ret-NH₂. An increased concentration of all-trans-retinol lowered the level of inhibition, as illustrated in FIG. 2B. This suggests that Ret-NH₂ and retinol compete for the same binding site(s). Dixon's and Lineweaver-Burk plots in the standard assay conditions yielded Kᵢ=0.1 μM for Ret-NH₂ when all-trans-retinol (K_{M}=0.3 μM) was assumed to be a substrate. HPLC analysis indicated that the amount of Ret-NH₂ at the start of the assay and at the end was unchanged (within 5%), and no intermediate other than non-inhibitory N-palmitoylretinamide was formed in the time course of the in vitro experiments. This estimated value reflects the very potent nature of the inhibition by 11-cis-Ret-NH₂.

To test the specificity of the inhibition, different isomers and derivatives of Ret-NH₂ were synthesized and tested. 11-cis-Ret-NH₂ (FIGS. 2C and D compound I) was the most potent inhibitor, while 9-cis-, 13-cis-, and all-trans-Ret-NH₂ had lower levels of potency with IC₅₀=640, 730, and 500 nM, respectively (FIG. 2C, D, compounds II, II, and IV). An acyclic analog of Ret-NH₂ had lower but comparable potency to 11-cis-Ret-NH₂ with 75% inhibition of isomerase at 10 μM concentration (FIG. 2 D, compound V). N-hydroxyretinylamine (reduced oxime, FIG. 2 D, compound VI) had higher IC₅₀. Interestingly, saturation of the C₁₃₋₁₄ double bond lowered the potency by about 10-fold for 11-cis- and all-trans-isomers, suggesting that the presence of the double bond in D-position of a protonated amine is important for the inhibition (FIG. 2 D, compounds VII and VIII). N-Alkylated derivatives of Ret-NH₂ failed to effectively inhibit isomerization, suggesting that these analogs do not fit optimally to the binding pocket of the isomerase (FIG. 2 D, compounds IX, X, and XI). Ret-NH₂ and its derivatives are not substrates for the isomerase. The inhibition was specific to the amino derivatives, as neither all-trans-thioretinol nor all-trans-13,14-dihydroretinol influenced the isomerization.

FIG. 2 shows inhibition of 11-cis-retinol isomerase activity by Ret-NH₂ and its derivatives. A. 11-cis-Ret-NH₂ inhibits isomerase activity in low μM concentrations in vitro without affecting LRAT activity. Gray bars and white bars correspond to relative amounts of 11-cis-retinol and all-trans-retinyl esters, respectively, as a function of increased concentration of 11-cis-Ret-NH₂. B. Relation between initial reaction rate (v) and different concentrations of all-trans-retinol and 11-cis-Ret-NH₂. The plotted graph indicates that inhibition of isomerase by Ret-NH₂ is reversible and can be competed out by all-trans-retinol. C. Potency of the isomerase inhibition by different Ret-NH₂ isomers. D. Inhibition of 11-cis-retinol production in the isomerase assay by Ret-NH₂ analogs.

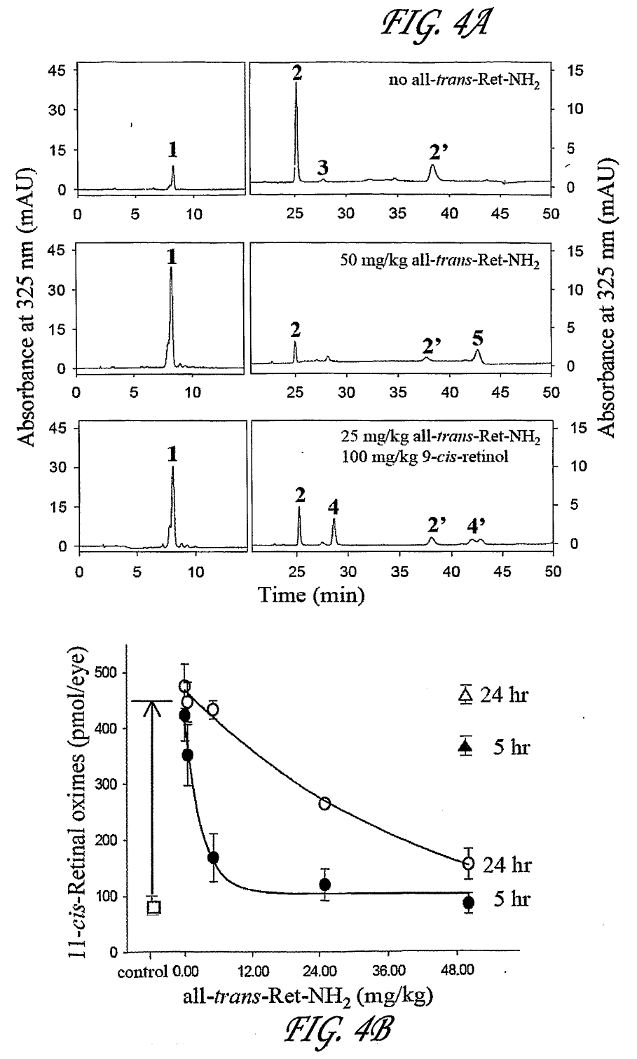

The specificity of Ret-NH₂ was further tested by biochemical assays. The experiments showed that the inhibitor binds to RPE protein(s), and it does not bind to the most abundant protein of the RPE, RPE65. When the RPE microsomes were solubilized with DHPC, the isomerase activity decreased only slightly. Then [³H]-Ret-NH₂ was added to the RPE extract and loaded on a gel filtration column. Proteins were fractionated and eluted between fractions 18 and 60 (FIG. 3A), while the radioactivity was eluted in fractions 36-38. RPE65 was identified by immunoblotting in fractions 23-34 (FIG. 3B). The most abundant proteins in this fraction were identified by mass spectrometry and include glucose phosphate isomerase, D-glyceraldehyde-3-phosphate dehydrogenase, phosphoglycerate mutase, triose phosphate isomerase, enolase, annexin V, calbindin 2, and LRAT. Identification of less abundant proteins is in progress. This radioactive elution profile suggests that [³H]-Ret-NH₂ formed a complex with a protein(s) different from RPE65. LRAT eluted in most fractions of the chromatogram, suggesting that multiple oligomeric protein-detergent complexes were present in the extract (FIG. 3B). In control experiments, retinoids that weakly inhibit the isomerization reaction, such as retinoic acid, all-trans-retinol, and Ret-NH₂ acetyl amide (XIII), eluted either in the void volume or with low molecular weight compounds. However, a potent inhibitor, an acyclic analog of Ret-NH₂ (V), eluted in fractions 36-38 similarly to Ret-NH₂. In control experiments using heat-denaturated proteins, [³H]-Ret-NH₂ eluted in fractions 48-60, suggesting that specific binding requires non-denatured proteins. In direct assays, Ret-NH₂ did not inhibited retinol dehydrogenases or retinyl acetate hydrolase (Kuksa et al., Vision Res 43:2959-81, 2003) present in the RPE.

FIG. 3 shows gel filtration chromatography of RPE proteins. RPE microsomes were solubilized with 10 mM DHPC, incubated with 11,12-di[³H]-all-trans-Ret-NH₂ (1 μM), and loaded on a Superdex 200 column equilibrated with Tris/HCl buffer, pH 7.5, containing 4 mM DHPC. Proteins were eluted with a constant flow rate of 0.4 ml/min. The protein and radioactivity levels of 0.4 ml fractions were examined by SDS-PAGE and scintillation counting (A), and immunoblot with anti-RPE65 and anti-LRAT antibodies (B). The star indicates the fraction associated with maximum radioactivity.

### Example 2

### Retinylamine Affects Recovery of the Chromophore and Visual Functions in Treated Mice

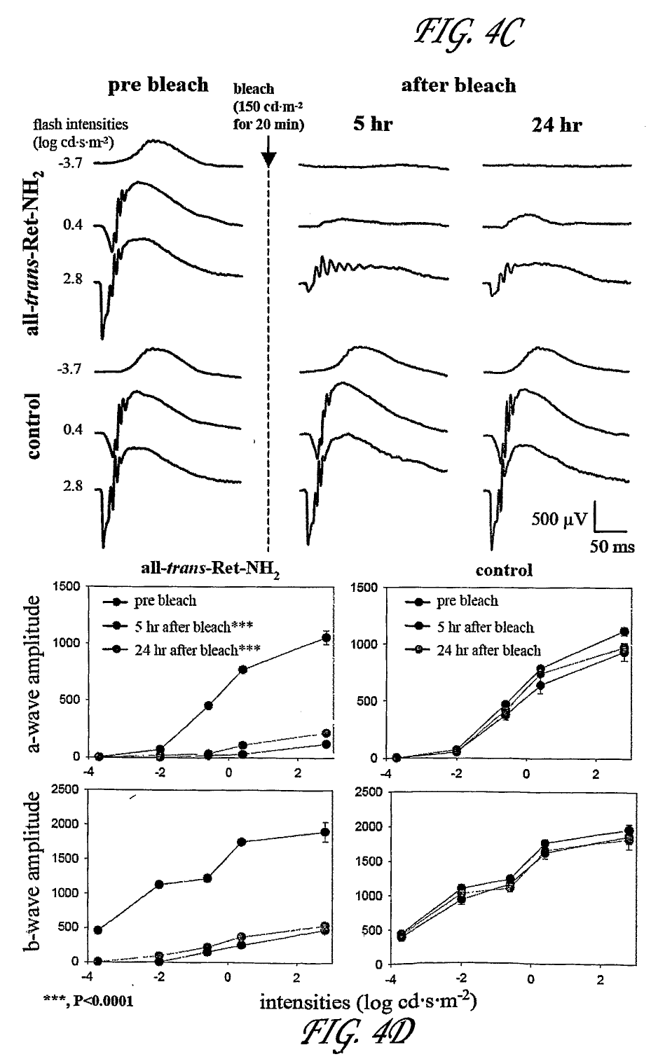

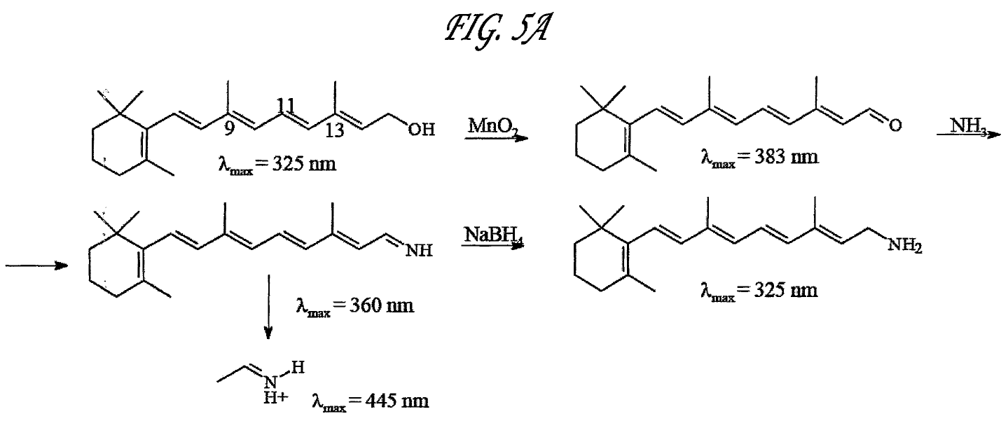

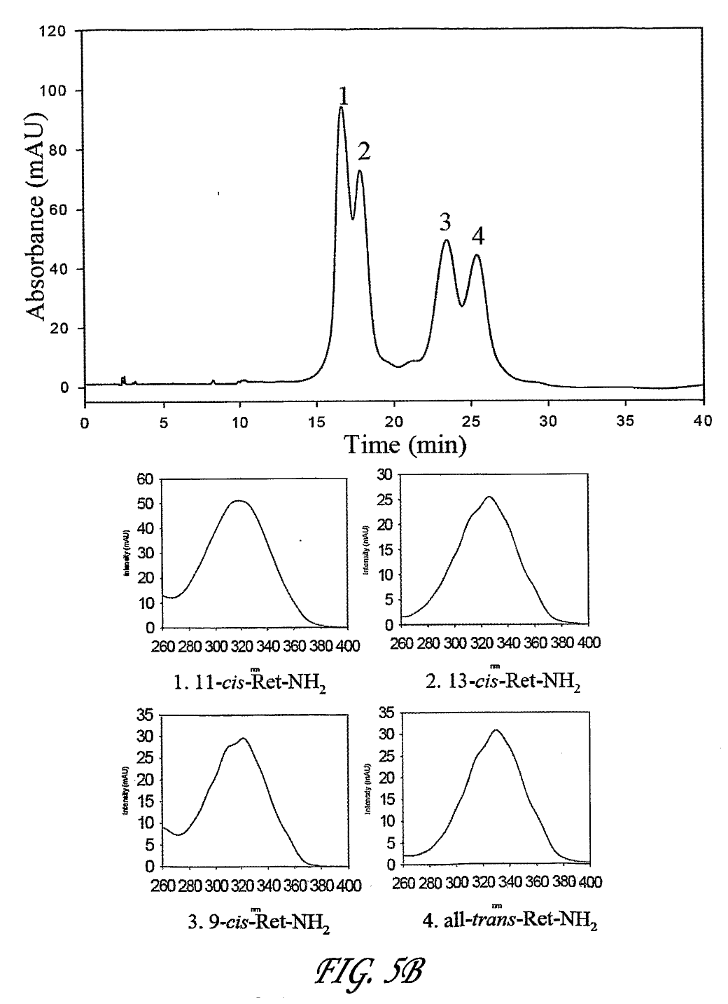

Retinylamine (Ret-NH₂) specifically inhibits regeneration of the 11-cis-retinal in mice following exposure to intense illumination. In this experiment all-trans-Ret-NH₂ was chosen as an inhibitor since it is more stable and more available than the 11-cis-isomer. Mice were gavaged with all-trains-Ret-NH₂, exposed to intense light for 20 min, and then allowed to recover in the dark for 5 hours. The total amount of different forms of retinoids in all experiments was unchanged. In treated mice only a residual amount of the 11-cis-retinal was present (peak 2 and 2′, FIG. 4A, middle panel), while untreated mice 11-cis-retinal recovered completely (peak 2 and 2′, FIG. 4A, top panel). Levels of all-trans-retinyl esters were elevated in treated animals (FIG. 4A, peak i, middle panel) as compared with controls, indicating that LRAT activity is not affected in vivo, in accordance with our in vitro results. Treatment with all-trans-Ret-NH₂ did not affect the uptake, transport or oxidation of 9-cis-retinol in treated animals (FIG. 4A, third panel). For this experiment mice were treated with both 9-cis-retinol and all-trans-Ret-NH₂, and bleached after 24 hours, and were allowed to recover for 5 hours in the dark. The presence of 9-cis-retinal was confirmed in treated animals (see peaks 4 and 4′ in FIG. 4A bottom panel, corresponding to syn- and anti-9-cis-retinal oximes, respectively). 11-cis-Retinal was not reduced and/or esterified after treatment with Ret-NH₂. This suggests that all-trans-Ret-NH₂ did not compete with the chromophore, as expected, for the binding pocket of rhodopsin, oxidation/reduction by the RPE, or photoreceptor dehydrogenases or transport to and from the RPE. This supports the idea that Ret-NH₂ is a specific inhibitor of the isomerization reaction.

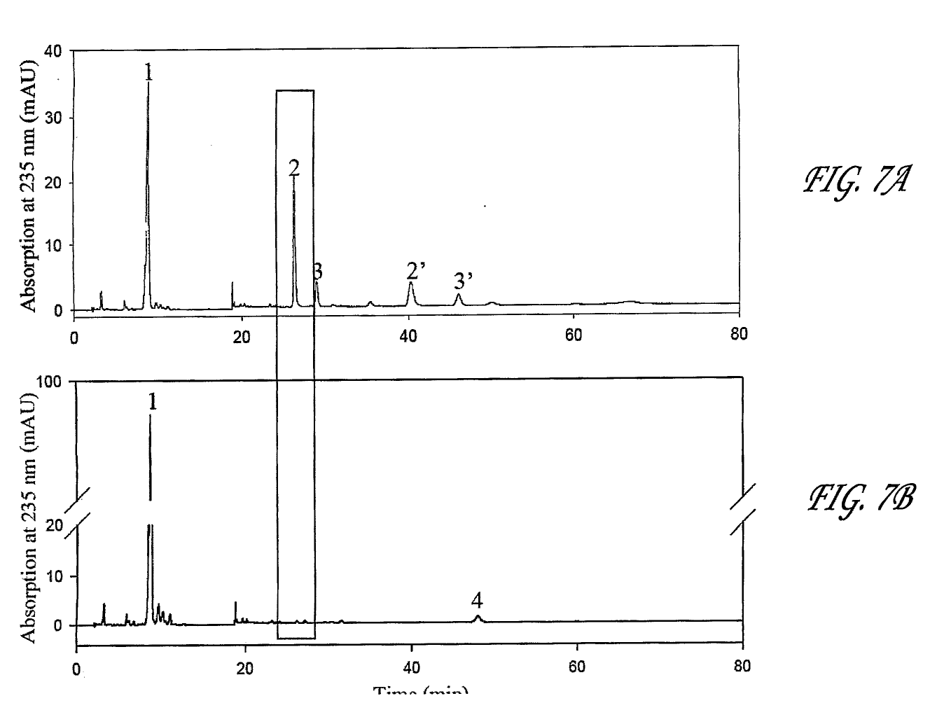

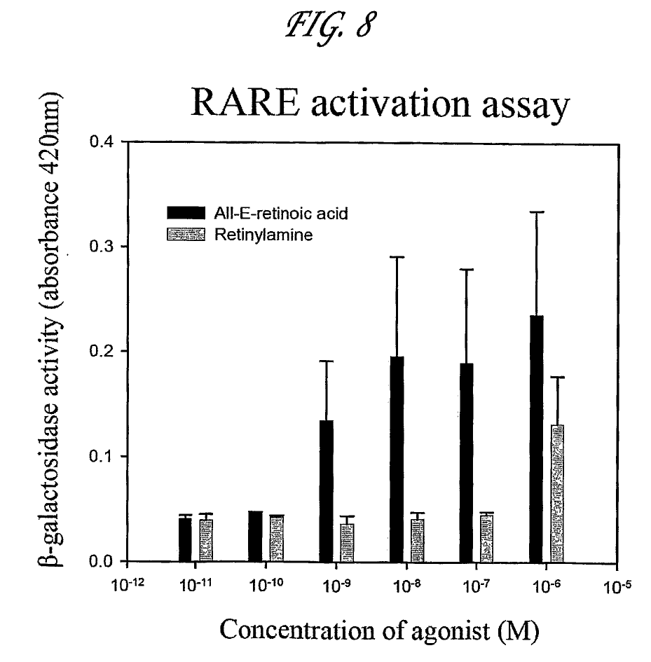

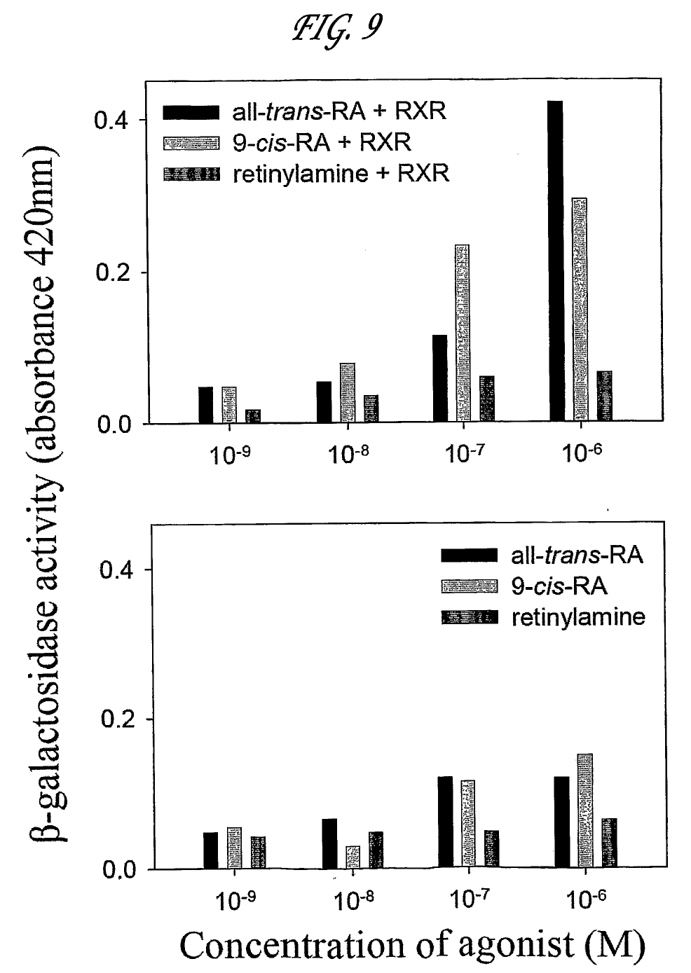

The 5 and 24 hour dose-dependencies of chromophore recovery after Ret-NH₂ treatment and a 20 min high-intensity light exposure are shown in FIG. 4B. Ret-NH₂-treated animals recovered the chromophore much more slowly than untreated animals (IC₅₀=2 mg/kg for 5 hours recovery, FIG. 4B, filled circle). The effect of Ret-NH₂ on chromophore recovery was reversible which is demonstrated by the fact that the level of chromophore increased after mice were kept in the dark for 24 hours compared with 5 hours (IC₅₀=24 mg/kg for 24 hours recovery, FIG. 4B open circle). Mice treated with all-trans-Ret-NH₂ (50 mg/kg) and not exposed to light had normal levels of retinoids in the eye after 30 hours. 13-cis-RA was shown to inhibit the retinoid cycle in vitro as well as in treated animals. Radu, et al., Proc Natl Acad Sci USA 100, 4742-7, 2003; Sieving, et al., Proc Natl Acad Sci USA 98, 1835-40, 2001. The potency of Ret-NH₂ was tested in parallel with 13-cis-RA. In the conditions of our assay, 13-cis-RA (50 mg/kg) was ineffective (FIG. 4B, red triangles). Ret-NH₂ completely blocks recovery of chromophore after all of photoreceptor 11-cis-retinal is photoisomerized to all-trans-by longer light treatment (FIG. 7). Thus, Ret-NH₂ is more specific and very potent inhibitor of the isomerization reaction compared with 13-cis-RA.

FIG. 4 shows retinylamine inhibits regeneration of vision chromophore in vivo. A. Chromatographic separation of nonpolar retinoids from WT mouse eyes. Mice were gavaged with all-trans-Ret-NH₂ 24 hours prior to bleaching for 20 min at 150 cd·m⁻² W in the Ganzfeld chamber. Regeneration of 11-cis-retinal was allowed for 24 hours in the dark, and retinoids were extracted from the eye and separated by normal phase HPLC as described in Materials and Methods. The peaks were identified based on elution time and absorption spectra correspond to the following retinoids: 1, all-trans-retinyl esters; 2,2′, syn- and anti-11-cis-retinal oximes; 3, syn-all-trans-retinal oxime; 4,4′, syn- and anti-9-cis-retinal oximes; 5, all-trans-retinol. B. Changes of 11-cis-retinal oxime levels at increasing doses of all-trans-Ret-NH₂ during 5 hours or 24 hours of dark adaptation. Square indicates the level of 11-cis-retinal oximes that was present in mice gavaged with vegetable oil without retinoids just after bleaching, and arrow indicates the increase of visual chromophore during 5 hours of dark adaptation. Triangles correspond to the level of 11-cis-retinal found in the mice gavaged with 50 mg/kg of 13-cis-RA. C and D. Single flash ERG responses of increasing intensity for all-trans-Ret-NH₂ treated mice and control mice. Serial responses to increasing flash stimuli were obtained for all-trans-Ret-NH₂ treated and control mice for selected intensities (C) and plotted as a function of a-wave and b-wave versus varying light intensities under dark-adapted conditions (D) before bleach and at 5 hours and 24 hours after bleach. The dark-adapted mice were bleached with intense constant illumination (150 cd·m⁻²) for 20 min. The responses from all-trans-Ret-NH₂ treated mice were significantly attenuated by single dose administration (50 mg/kg) compared with control mice (P<0.0001, one-way ANOVA). SE bars are shown.

FIG. 7 shows single flash ERG responses of increasing intensity for all-trans-Ret-NH₂ treated mice and control mice in light-adapted conditions. Serial responses to increasing flash stimuli were obtained for all-trans-Ret-NH₂ treated and control mice for selected intensities (A) and plotted as a function of a-wave and b-wave versus light intensities under light-adapted conditions (B) before and at 5 hours and 24 hours after bleach with intense constant illumination (150 cd·m₋₂) for 20 min. The responses from all-trans-Ret-NH₂ treated mice were attenuated weakly by single dose administration (50 mg/kg) compared with control mice (p>0.1, one-way ANOVA). SE bars are shown.

The conclusions derived from the analysis of retinoids of Ret-NH₂-treated mice were supported by ERG analysis of visual functions. ERG responses were significantly affected by a decrease in 11-cis-retinal regeneration with all-trans-Ret-NH₂ administration. Single doses of increasing concentrations of all-trans-Ret-NH₂ (0.5 to 100 mg/kg) were delivered to mice via oral gavage. Treated animals did not exhibit symptoms of systemic toxicity such as weight loss or gastrointestinal disorder. Next, the impact of all-trans-Ret-NH₂ administration on the visual physiology of wild-type mice was evaluated in vivo with ERGs. Dark-adapted ERGs were performed serially 24 hours after all-trans-Ret-NH₂ gavage (50 mg/kg) and 5 and 24 hours after intense bleaching. Treated mice showed normal waveforms and responses for recordings obtained before bleach. However, after 20 min constant illumination and 5 hours dark adaptation a- and b-wave amplitudes from treated mice were significantly attenuated, and they remained so even after 24 hours (P<0.0001, one-way ANOVA). In contrast, the dark-adapted state of control mice was fully recovered 5 hours after bleach (FIGS. 4C and D). The cone function was tested using photopic ERG conditions. Recovery of cone function was not affected by all-trans-Ret-NH₂ to the same extent as rod function. This could be a result of the fact that cones are able to recover chromophore faster than rods and do not saturate even after intense bleach, allowing them to operate at low levels of regenerated cone pigments (FIG. 6).

FIG. 6 shows retinylamine inhibits regeneration of vision chromophore in vivo after intense bleach. Chromatographic separation of nonpolar retinoids from WT mouse eyes. Mice were gavaged with 100 mg/kg of all-trans-Ret-NH₂ 24 hours prior to bleaching. Mice were next exposed to 500 cd m⁻² for 48 min bleaching in the Ganzfeld chamber. Regeneration of 11-cis-retinal was allowed for 24 hours in the dark, and retinoids were extracted from the eye and separated by normal phase HPLC as described in Materials and Methods. The peaks identified based on elution time and absorption spectra correspond to the following retinoids: 1, all-trans-retinyl esters; 2,2′, syn- and anti-1-cis-retinal oximes; 3,3′ syn- and anti-all-trans-retinal oximes; 4, all-trans-retinol.

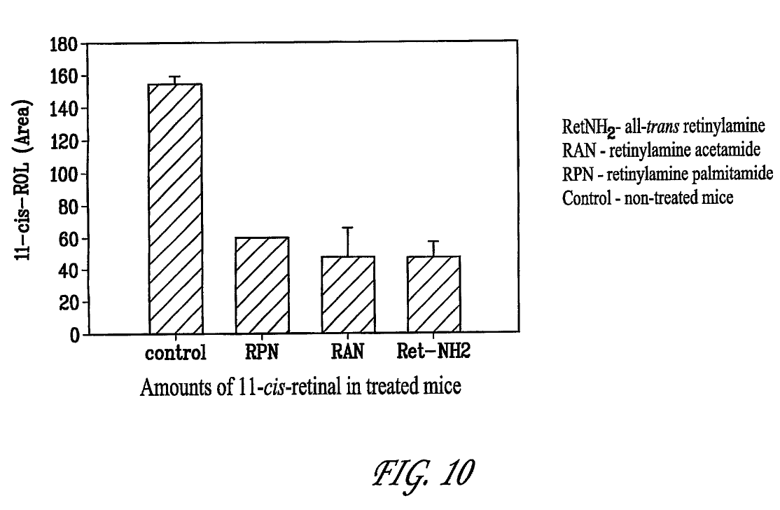

13-cis-RA can isomerize to all-trans-RA and then to 9-cis-RA, both of which are potent ligands of the RA receptor (RAR). 9-cis-RA also binds and activates the retinoid X receptor (RXR). This contributes to the toxicity of the RAR and RXR ligands. It was investigated whether Ret-NH₂ can activate these nuclear receptor using reporter-cells as described previously (20). A β-galactosidase activity assay was employed to examine if all-trans-RA, 9-cis-RA, and Ret-NH₂ are agonists for these nuclear receptors. In contrast to all-trans-RA and 9-cis-RA, all-trans-Ret-NH₂ does not activate protein transcription at micromolar concentrations through either RAR or RXR (FIGS. 8 and 9).

FIG. 8 shows response of F9-RARE-lacZ reporter cell line to retinylamine. F9-RARE-lacZ cells express endogenous RAR and RXR and were transfected with a construct of lacZ under the control of a minimal promoter and upstream DR5 elements (1). F9-RARE-lacZ cells were treated with different doses of all-trans-RA or Ret-NH₂ for 24 hours after which the cells were harvested and the β-galactosidase activity was measured using the soluble substrate o-nitrophenyl β-D-galactopyranoside. The colorless substrate was cleaved by β-galactosidase to yellow colored o-nitrophenol, whose absorbance was measured at 420 nm using a spectrophotometer. The experiment was repeated twice with similar results. The RARE reporter cell line F9-RARE-lacZ (SIL15-RA) was a kind gift from Dr. Michael Wagner (SUNY Downstate Medical Center) and Dr. Peter McCaffery (University of Massachusetts Medical School). The RA-responsive F9 cell line was transfected with a reporter construct of a RARE element derived from the human RA receptor-β gene (RARβ) placed upstream of the E. coli lacZ gene (1). Cells were grown in L15-CO₂ media containing N3 supplements and antibiotics. Cells were stimulated for 24 hours in the dark at 37° C. and 100% humidity with RA or Ret-NH₂ dissolved in EtOH at the indicated concentrations, lysed, and assayed for the expression of β-galactosidase using the β-galactosidase Enzyme Assay System (Promega, Madison Wis.). Wagner, M., Han, B. & Jessell, T. M. (1992) Development 116, 55-66, incorporated herein by reference.

FIG. 9 shows activation of DR1-elements by retinylamine. HEK-293 cells were transfected with a construct of lacZ under the control of a minimal promoter and five consecutive upstream DR1 elements. (Top) HEK-293 cells were cotransfected with both DR1-reporter construct and mouse RXRA under the control of the CMV promoter. The cells were then treated with indicated levels of all-trans-RA, 9-cis-RA, or Ret-NH₂ for 48 hours. The cells were harvested and β-galactosidase activity was assayed as described in Materials and Methods. (Bottom) DR1-reporter transfected cells were treated with different doses of all-trans-RA, 9-cis-RA, or Ret-NH₂ in the absence of RXR for 48 hours. The cells were harvested and β-galactosidase activity was assayed as described in Materials and Methods. Mouse RXR-A was cloned using the primers 3′-GGGCATGAGTTAGTCGCAGA-5′ and 3′-AGCTGAGCAGCTGTGTCCA-5′ from reverse transcribed mouse liver cDNA. The RXR-A ORF was then subcloned into pcDNA3.1 Directional TOPO vector (Invitrogen, Carlsbad Calif.) using the primers 3′-CACCATGGACACCAAACATTTCCT-5′ and 3′-AGCTGAGCAGCTGTGTCCA-5′. The RXRE element from the vector RXR (2) Translucent reporter vector (Panomics, Redwood City, Calif.) was amplified using the primers 3′-CTCAACCCTATCTCGGTCTATTCT-5′ and 3′-ATGCCAGCTTCATTATATACCCA-5′ and cloned upstream of the minimal promoter and β-galactosidase of pBLUE-TOPO (Invitrogen). This places five consecutive DR1 elements upstream of β-galactosidase, the expression of which becomes dependent on activation of RXR and formation of RXR homodimers. Both strands of all constructs were sequenced to ensure that no mutations were present. HEK-293 cells were transiently transfected using Lipofectamine 2000 (Invitrogen) according to the manufacturer's protocol and then 24 hours later split into 24 well plates to ensure an equal number of transfected cells in each assay well. Cells were stimulated with appropriate concentrations of RA, 9-cis-RA, or Ret-NH₂. The expression of β-galactosidase was assayed 48 hours later as described above.

Next, the RA oxidation pathway was investigated. Using HEK-293 cells transfected with CYP26A1 cDNA RA was found to readily oxidized to 4-oxo-, 4-hydroxy-, and 18-hydroxy-metabolites, whereas there was no evidence of the presence of hydroxy- or oxo-Ret-NH₂. This observation suggests that Ret-NH₂ is not directly metabolized through the pathway of cytochrome P450 CYP26 enzymes. In addition to secretion, Ret-NH₂ might be degraded first by deamination to all-trans-retinol/retinal, which was seen to increase in Ret-NH₂ treated cells. In addition to secretion, Ret-NH₂ is amidated to N-retinylpalmitamide, as confirmed by HPLC and mass spectrometry analysis of the liver samples of treated mice and chemical synthesis of standards. N-Retinylpalmitamide (XII) did not inhibit isomerization, but this amide and N-retinylacetamide (XI) were also potent inhibitors of regeneration in mice. These observations explain the long-lasting effect of RetNH₂, as the amide is stored and hydrolyzed back to the free amine.

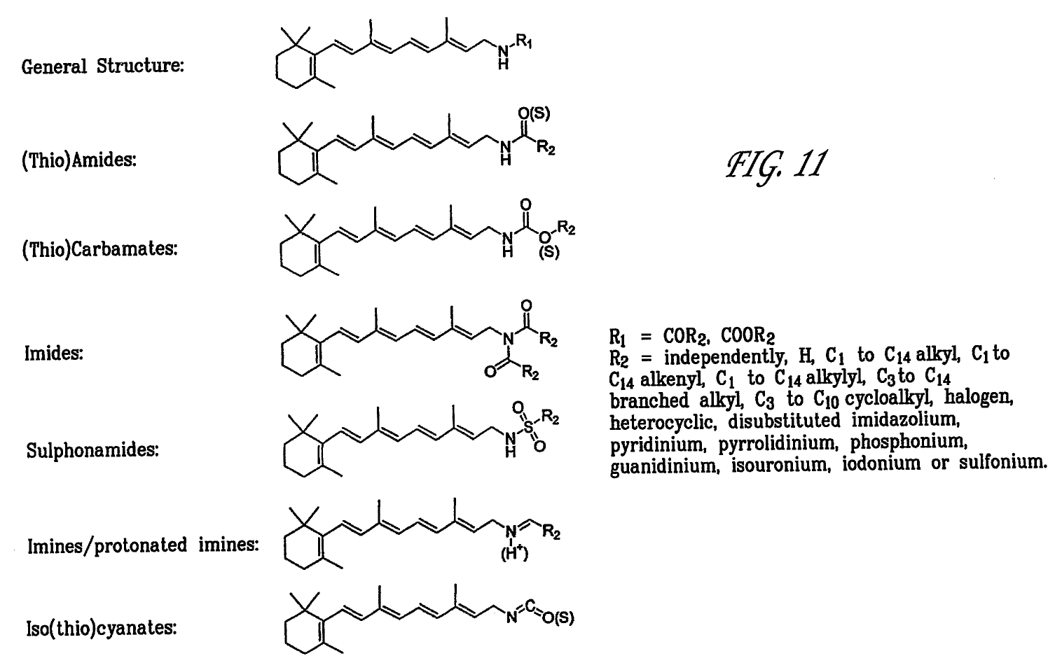

FIG. 10 shows retinylamides inhibit regeneration of vision chromophore in mice following treatment with retinylamide and shows that treating mice by gavage with retinylamides has an effect by reducing the amounts of free retinals as in the case of free retinylamine. FIG. 10 shows the relative amounts of 11-cis-retinal in the mouse eyes after treating with an inhibitor and light bleaching. Mice were gavaged with the solution of inhibitor in vegetable oil (control—non-treated, RPN—retinylpalmitamide, RAN—retinylacetamide, Ret—NH₂-all-trans-retinylamine), kept in dark for 16 hours, then light stimulated and kept in dark for additional 5 hours before their eyes were analyzed. The effect of amides is same as free retinylamine.

### Example 3

### A New Tool to Study the Mechanism of all-trans-retinol Isomerization and the Pathogenesis of Retinal Disease

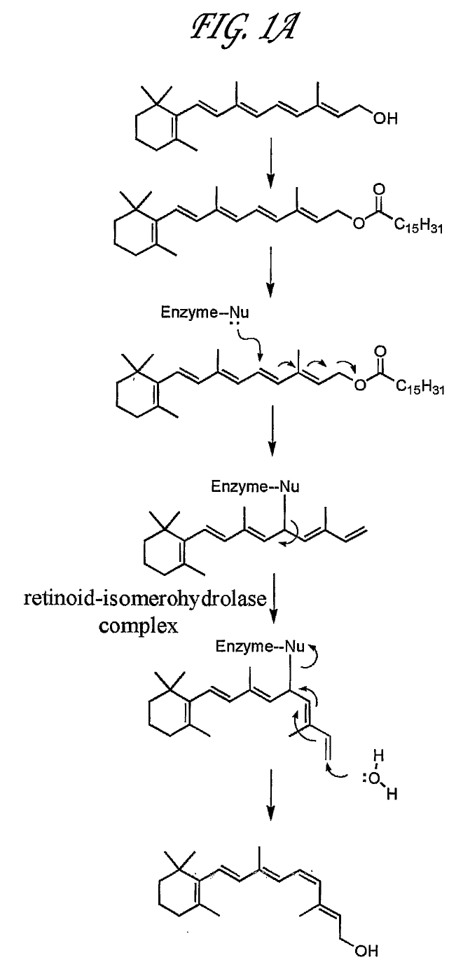

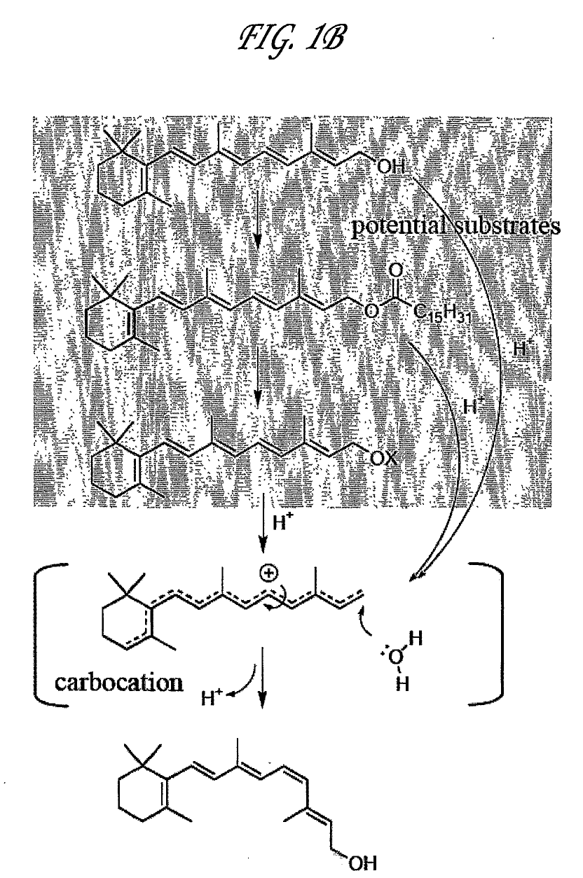

In the course of studies of the isomerization mechanism a specific and potent inhibitor of the key enzyme of the retinoid cycle was uncovered (McBee et al., Prog Retin Eye Res 20:469-52, 2001). The enzyme that carries out the isomerization reaction is not known, but a few different theories have emerged with regard to the mechanism of isomerization. In one mechanism all-trans-retinyl esters are the direct substrate of the isomerohydrolase (Rando, Biochemistry 30:595-602, 1990), a hypothetical enzyme that couples the energy of ester hydrolysis with the endothermic isomerization of the C₁₁₋₁₂ double bond (FIG. 1A). A number of observations on the isomerization reaction have brought into question the isomerohydrolase hypothesis (Kuksa et al., Vision Res 43:2959-81, 2003). To reconcile these discrepancies, a second mechanism was proposed, which offers a different set of answers as well as questions regarding the isomerization step. In the proposed mechanism (FIG. 1B), an as yet unidentified intermediate undergoes protonation at the oxygen atom that leads ultimately to an elimination of the carboxylic acid, leaving all-trans-retinol as a retinyl carbocation. The positive charge is delocalized throughout the conjugated double bonds (FIG. 1B). Results presented here suggest that Ret-NH₃⁺ might mimic the transition state analog in the carbocation mechanism. Because 11-cis-Ret-NH₂ is a more potent inhibitor than all-trans- it may be that the retinyl carbocation-like structures resemble the 11-cis-retinal configuration.

Several observations from our in vitro and in vivo experiments indicate that Ret-NH₂ is a specific inhibitor of the isomerization process. In vitro assays demonstrate that this compound does not inhibit LRAT, retinol dehydrogenases, or retinyl esters hydrolases. Moreover, Ret-NH₂ does not bind to CRALBP or RPE65. The most potent inhibitor was 11-cis-Ret-NH₂, and modifications of the amino group (with the exception of N-retinylhydroxylamine) decreased the potency, suggesting a tight fit of the compound to the active site. In the case of N-retinylhydroxylamine there could be a hydrogen bond network due to the substitution of an —NHOH group for the —NH₃⁺ group, and this compound could be protonated in the binding site of the enzyme. Most of the tested retinoids are protonated at neutral pH, and this feature appears to be a prerequisite for potent inhibition. Bulky Ret-NH₂ derivatives such as N-alkyl-Ret-NH₂ are not good inhibitors and most likely do not fit well into the binding pocket of the isomerase. Based on this observation, one may speculate that the real substrate is not a bulky hydrophobic retinyl ester, but presumably a more polar and less substituted component, for example, a retinol or a low molecular weight retinyl ester. In vivo results also support the idea that no other step in the retinoid cycle is affected by Ret-NH₂ except the isomerization reaction.

Inhibition of the retinoid cycle and Stargardt's disease—Mutation in the ABCR gene was associated with recessive Stargardt macular dystrophy (Allikmets et al., Nat Genet. 15:236-46, 1997). Mata and colleagues found that A2E, the major fluorophore of lipofuscin, were ˜four-fold more abundant in 8-month-old Abcr+/− mice and ten times more abundant in Abcr−/− than in wild-type mice (Mata et al., Invest Opthalmol Vis Sci 42:1685-90, 2001). This accumulation was strongly dependent on light exposure. It is speculated that this accumulation may be responsible for the photoreceptor death and severe vision loss in Stargardt's patients (Weng et al., Cell 98:13-23, 1999). Based on these findings, a new therapeutic strategy was proposed to inhibit lipofuscin accumulation (thus slowing down the retinoid cycle) in a mouse model of Stargardt's disease. For this purpose 13-cis-RA (Isotretinoin, or Accutane®), which inhibits 11-cis-RDH (Radu et al., Proc Natl Acad Sci USA 100:4742-7, 2003) and is associated with induced night blindness, has been used to slow the synthesis of 11-cis-retinal through the inhibition of 11-cis-RDH. Others have proposed that 13-cis-RA works to prevent chromophore regeneration by binding RPE65, a protein essential for the isomerization process in the eye (Law and Rando, Biochem Biophys Res Commun 161:825-9, 1989). It should be noted that the action of 13-cis-RA could still be different from the role previously proposed. Nonetheless, these investigators found that 13-cis-RA blocked the formation of A2E, and suggested that this treatment may inhibit lipofuscin accumulation and thus delay either the onset of visual loss in Stargardt's patients or the macular degeneration associated with lipofuscin accumulation. The proposed treatment is intriguing, but one must be aware of a potential problem associated with blocking the retinoid cycle and the formation of unliganded opsin (Van Hooser et al., J Biol Chem 277:19173-82, 2002; Woodruff et al., Nat Genet. 35:158-164, 2003). This may result in more severe consequences and worsening of the patient's prognosis. Failure of the chromophore to form may lead to progressive retinal degeneration and in an extreme case will produce phenotype similar to LCA.

Comparison of Ret-NH₂ to 13-cis-RA—The chemistry and biological activity of the inhibitors 13-cis-RA and Ret-NH₂ stand in sharp contrast with each other. In the case of 13-cis-RA, its two possible metabolic fates are isomerization to all-trans-isomer or oxidation, glucuronidation, and secretion (Li et al., J Chromatogr B Biomed Appl 683:155-62, 1996). 13-cis-RA will occur in equilibrium with all-trans-RA, which can activate the RA-dependent transcriptional pathway. RA was shown to activate transcription of target genes by binding to nuclear RA receptors (RAR) and retinoid X receptors (RXR) (Chambo, Faseb J 10:940-54, 1996). Particularly, 13-cis-RA is highly toxic during pregnancy (Mitchell and Van Bennekom, J Am Acad Dermatol 49:1201-2, 2003). There are three different isotypes of RAR (α, β and γ) coded by different genes. The ligand-binding domain of the three RARs is highly conserved and binds both 9-cis-RA and all-trans-RA. The ligand-binding domain of the three isotypes of RXR (α, β and γ) is also conserved and binds only 9-cis-RA. RAR mediates activation of genes containing cis-acting response elements (RARE) by forming RAR/RXR heterodimers. RARE elements consist of direct repeats of hexameric motifs PuG(G/T)TCA separated by 1-5 bases and have been found in the promoter region of many genes including the RARβ gene (Sucov et al., Proc Natl Acad Sci USA 87:5392, 1990). RXR homodimers can be activated by 9-cis-RA (Heyman et al., Cell 68, 397-406, 1992) and other hydrophobic substances. RXR homodimers are specific for DR1 elements of hexameric motifs separated by a single base pair as found in the CRBP II promoter (Goldstein et al., Arch Biochem Biophys 420, 185-93, 2003). The activation of genes mediated by RAR or RXR can be studied using reporter genes containing lacZ under the control of a minimal promoter and the appropriate DR element located immediately upstream. In our assay, as expected, 9-cis- and all-trans-RA activated transcription of DR1 or DR5 RARE elements, while Ret-NH₂ did not. This suggests that Ret-NH₂ should be a safer alternative to RA and RA-based pharmacological inhibitors such as 13-cis-RA whose toxicity makes them unsuitable in many patients.

Much less is known about the pharmacokinetics of Ret-NH₂ in vivo. Ret-NH₂s cannot be stored in the ester form. Whether they undergo amidation or deamination is an open question and requires further investigation. As mentioned here, Ret-NH₂ is not readily metabolized by oxidative hydroxylation in reactions catalyzed by Cyp26 (Abu-Abed et al., Genes Dev 15:226-40, 2001). In addition, Ret-NH₂ can be stored in the form of amide and does not undergo oxidative hydroxylation by Cyp26s. The amide storage form is reversible with free amine, explaining the long-lasting inhibition of isomerization by Ret-NH₂ in mice. More importantly, Ret-NH₂ does not activate the RAR and RXR nuclear receptors. Thus, it is reasonable to speculate that higher potency per dose, lower toxicity, and preservation of the cone vision makes Ret-NH₂ a highly improved alternative to 13-cis-RA.

Value of a specific inhibitor of isomerization for understanding of the retinoid cycle—Identification of such a potent group of inhibitors will expand the studies of the retinoid cycle in vivo and in vitro. It now appears to be a relatively straightforward procedure to prepare tagged 11-cis-retinal in order to track its metabolism in the eye. These types of approaches together with two-photon microscopy (Imanishi et al., J Cell Biol 164:373-8, 2004) will allow us to study the dynamic flux of retinoids through the visual cycle in wild-type and genetically engineered mice. These inhibitors also appear to be useful ligands for affinity chromatography during efforts to isolate the isomerization complex.

### Example 4

### Experimental Procedures

Animals—All animal experiments employed procedures approved by the University of Washington Animal Care Committees and conformed to recommendations of the American Veterinary Medical Association Panel on Euthanasia and recommendations of the Association of Research for Vision and Ophthalmology. Typically, 6-8 week-old mice were used in all experiments.

Materials—Fresh bovine eyes were obtained from a local slaughterhouse (Schenk Packing Co., Inc., Stanwood, Wash.). Preparation of bovine RPE microsomes was performed according to previously described methods (Stecher et al., J Biol Chem 274:8577-85, 1999). All chemicals were purchased from Sigma-Aldrich (St. Louis, Mo.). 11-cis-Retinal was obtained from Dr. Rosalie Crouch (Charleston, S.C.).

Retinoid preparations—All-trans-retinol was obtained by reduction of all-trans-retinal with an excess of NaBH₄ in EtOH at 0° C. and purified by normal phase HPLC (Beckman Ultrasphere Si 5μ4.5×250 mm, 10% EtOAc/hexane; detection at 325 nm). Purified all-trans-retinol was dried under a stream of argon and dissolved in DMF to a final concentration of 3 mM and stored at −80° C. Retinoid concentrations in EtOH were determined spectrophotometrically. Absorption coefficients for Ret-NH₂s were assumed to be equal to those of retinol isomers. Hubbard, et al., Methods in Enzymology 18: 615-653, 1971; Robeson, et al., J. Am. Chem. Soc 77: 4111-4119.

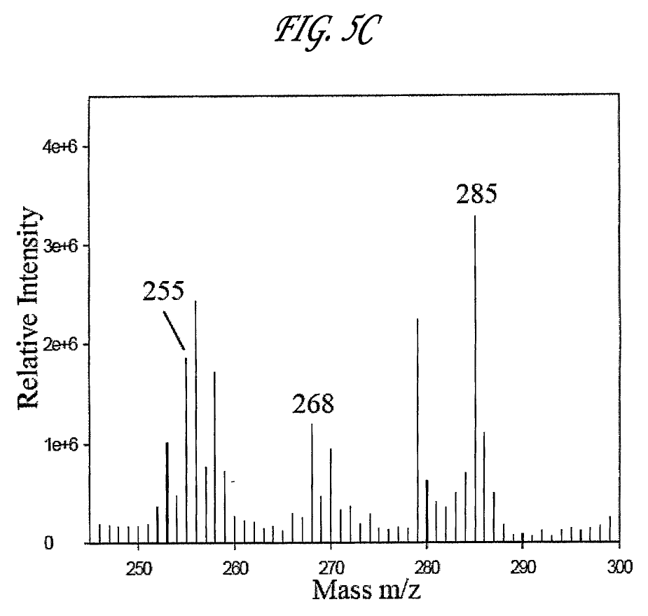

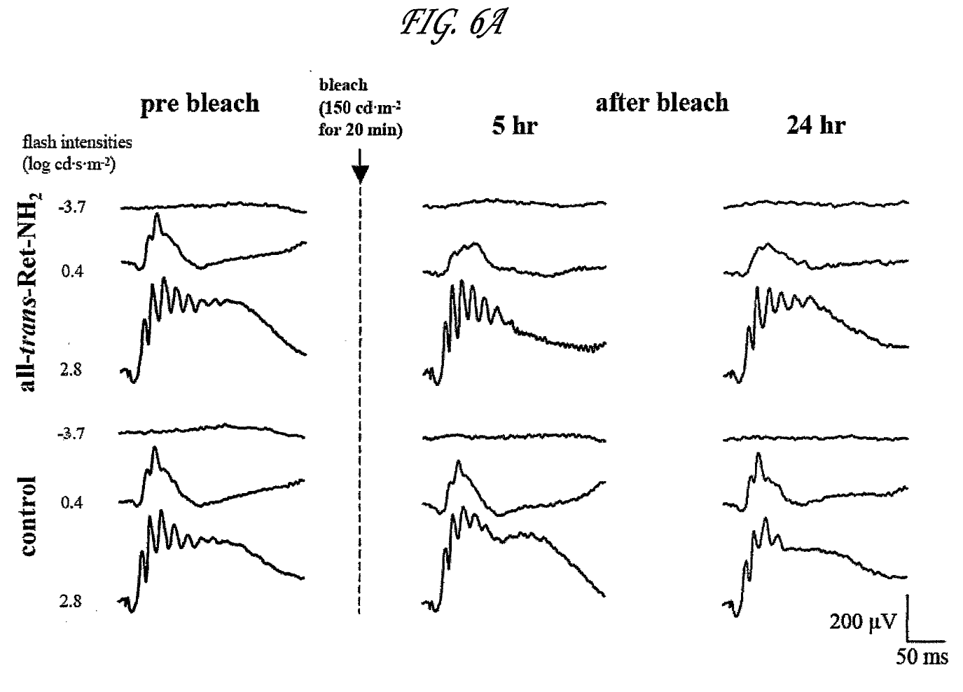

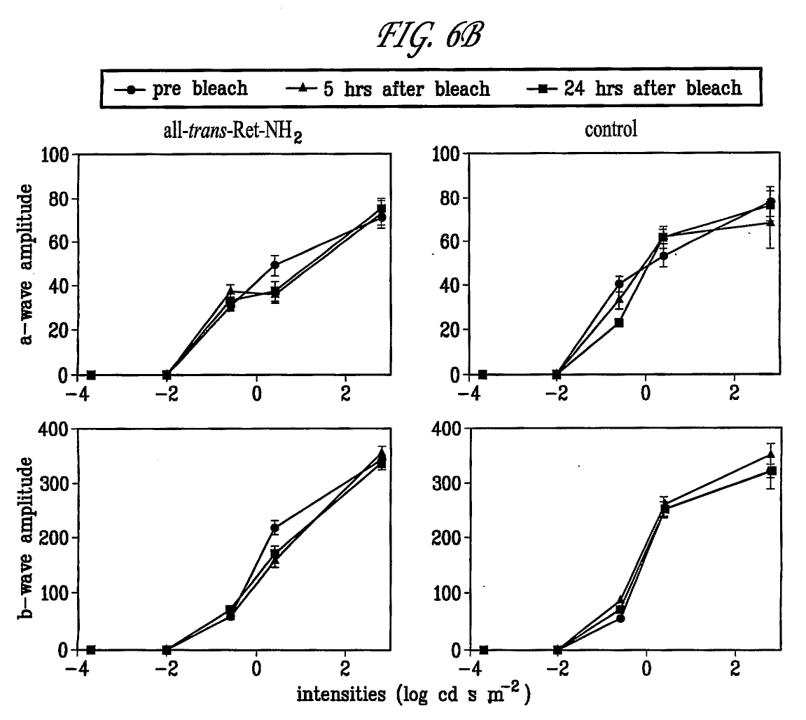

Chemical synthesis—Ret-NH₂ was obtained by a previously described method with some modifications. Yang et al., Proc Natl Acad Sci USA 94: 13559-64, 1997. Thus, the corresponding isomer of retinal was dissolved in EtOH and reacted with a 5-fold excess of 7 N NH₃ in MeOH for 1 hour at room temperature to form retinylimine. Then retinylimine was reduced to Ret-NH₂ with a 5-fold excess of NaBH₄. The reaction progress was followed spectrophotometrically. After 1 hour at 0° C., water was added and Ret-NH₂ was extracted twice with hexane. Combined hexane extracts were washed with water and brine, layers were separated, and the organic phase was loaded on a silica gel. The column was washed with hexane, then with 1:1 EtOAc/hexane. Ret-NH₂ was eluted with EtOAc with an addition of 10% 7 N NH₃/MeOH. The typical yield was 30% of pure Ret-NH₂ (FIG. 5). Prior to in vitro experiments, Ret-NH₂ was further purified using normal phase columns by elution with EtOAc/7 N NH₃ in MeOH (99:0.5).

FIG. 1 shows two proposed mechanisms of 11-cis-retinol formation. A. The first mechanism uses the energy of ester hydrolysis to drive the unfavorable isomerization of all-trans-isomer to its thermodynamically less stable 11-cis-isomer in a reaction catalyzed by the putative isomerohydrolase (Rando, Biochemistry 30:595-602, 1990). B. The second mechanism proposes the formation of 11-cis-retinol via a carbocation intermediate where all-trans-retinol, all-trans-retinyl ester, or another all-trans-retinoid derivative becomes protonated, followed by an elimination reaction yielding a retinyl carbocation. Hydration of the carbocation leads to the formation of 11-cis-retinol. The reaction is catalyzed by an unknown isomerase and energetically driven by mass action of binding proteins (Kuksa et al., Vision Res 43:2959-81, 2003).

FIG. 5 shows synthesis and HPLC separation of retinylamine isomers. (A) Ret-NH₂ was synthesized by oxidation of retinol to retinal with MnO₂ (shift of A_{λmax} from 325 to 383 nm). The oxidation product was further reacted with NH₃ in order to produce Ret-NH₂ (progress of the reaction was concomitant with blue shift of the absorbance maximum as well as significant red shift upon acidification). Retinylimine was reduced by NABH₄ to Ret-NH₂ (A_{λmax}=325 nm). Panel B represents the HPLC chromatogram of the isomers' separation (0.5% NH₂ in MeOH/EtOAc). The peaks were identified based on their absorbance maxima and shape of the spectra as follows: 1, 11-cis-; 2, 13-cis-; 9-cis-; all-trans-Ret-NH₂. Panel C shows the MS fragmentation patent or an-trans-Ret-NH₂ with the parent ion at 185 m/z and characteristic retinoid peaks at 268 and 255 m/z.

N-Substituted all-trans-Ret-NH₂s were prepared as described above, but instead of NH₃, an excess of the corresponding alkylamine was added to the solution of all-trans-retinal in EtOH. N-Alkyl-Ret-NH₂s were purified on an HPLC column using the conditions described above.

Hydroxylamine derivatives were prepared by the reaction of retinal with the corresponding hydroxylamines in EtOH. All-trans-retinal oximes were extracted with hexane, dried, redissolved in EtOH:MeOH (1:1) with an addition of acetic acid (10% v/v), and reduced with NaBH₃CN. MS analyses of synthesized retinoids were performed using a Kratos profile HV-3 direct probe mass spectrometer.

Retinyl amides were prepared by the reaction between all-trans-retinylamine and an excess of either acetic anhydride or palmitoyl chloride in anhydrous dichloromethane in the presence of N,N-dimethylaminopyridine at 0° C. for 30 min. After the reaction was complete, water was added and the product was extracted with hexane. The hexane layer was washed twice with water, dried with anhydrous magnesium sulfate, filtered, and evaporated. Mass analyses of synthesized retinoids were performed using a Kratos profile HV-3 direct probe mass spectrometer.

Reaction conditions for isomerase and LRAT reaction—The isomerase reaction was performed essentially as described previously (Stecher et al., J Biol Chem 274:8577-85, 1999). The reaction was carried out in 10 mM BTP buffer, pH 7.5, 1% BSA, containing 1 mM ATP and 6 μM apo-CRALBP. To investigate inhibition properties of Ret-NH₂ and its derivatives, RPE microsomes were preincubated for 5 min in 37° C. with the indicated compound in 10 mM BTP buffer, pH 7.5, 1% BSA, 1 mM ATP prior to addition of apo-CRALBP and all-trans-retinol. Ret-NH₂ and its derivatives were delivered to the reaction mixture in 1 μl of DMF, and the same volume of DMF was added to the control reaction. Each experiment was performed three times in duplicate. The average values were used and the standard deviations were calculated.

Mouse retinoid extraction and analysis—Retinoid analysis was performed under dim red light as described previously (Maeda et al., J. Neurochem 85:944-956, 2003; Van Hooser et al., J Biol Chem 277:19173-82, 2002). Mice were gavaged with retinoids as described previously (Van Hooser et al., J Biol Chem 277:19173-82, 2002).

Electroretinograms (ERGs)—Mice were prepared and ERG recording was performed as previously published (Haeseleer et al., Nat Neurosci 7:1079-87, 2004). Single flash stimuli had a range of measures (−3.7−2.8 log cd·s·m⁻²). Typically, three to four animals were used for the recording of each point in all conditions. Statistical analysis was carried out using the one-way ANOVA test.

Deigner et al., Science, 244: 968-971, 1989; Gollapalli et al., Biochim Biophys Acta. 1651: 93-101, 2003; Parish, et al., Proc. Natl. Acad. Sci. USA, 14609-14613, 1998; Radu, et al., Proc Natl Acad Sci USA, 101: 5928-5933, 2004.

When ranges are used herein for physical properties, such as molecular weight, or chemical properties, such as chemical formulae, all combinations and subcombinations of ranges and specific embodiments therein are intended to be included.

The disclosures of each patent, patent application and publication cited or described in this document are hereby incorporated herein by reference in their entirety.

Those skilled in the art will appreciate that numerous changes and modifications can be made to the embodiments of the invention and that such changes and modifications can be made without departing from the spirit of the invention. It is, therefore, intended that the appended claims cover all such equivalent variations as fall within the true spirit and scope of the invention.

What is claimed:

1. A method for treatment or prophylaxis of a degenerative disease in a vertebrate eye, comprising administering to the vertebrate an effective amount of a positively charged retinoid derivative in a pharmaceutically or ophthamologically acceptable vehicle.

2. The method of claim 1, wherein the positively charged retinoid derivative is a retinylamine derivative.

3. The method of claim 1, wherein the positively charged retinoid derivative inhibits an isomerization step of the retinoid cycle.

4. The method of claim 1, wherein the positively-charged retinoid derivative is a retinoid derivative of formula I: 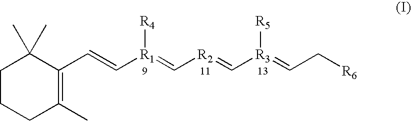
or a stereoisomer, prodrug, pharmaceutically or ophthamologically acceptable salt, hydrate, solvate, acid salt hydrate, N-oxide or isomorphic crystalline form thereof,
wherein at least one of R₁, R₂, R₃, R₄, R₅ or R₆ is a primary, secondary, tertiary or quaternary amine;
wherein R₄ or R₅ are, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₇R₈⁺X⁻, CH₂—NR₇R₈, NR₇R₈, or NR₇R₈R₉⁺X⁻;
wherein R₆ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₇R₈⁺X⁻, OR₇, SR₇, CH₂—NR₇R₈, NR₇R₈, or NR₇R₈R₉⁺X⁻;
wherein R₇, R₈, and R₉ are independently, H, C₁ to C₆ alkyl, C₂ to C₆ alkenyl, C₂ to C₆ alkynyl, or C₃ to C₄ cycloalkyl, OH, or OR₁₀, wherein R₁₀ is C₁ to C₆ alkyl; and X is an anion, Cl, Br, I, SO₃H, or P(O)₂(OH)₂.

5. The method of claim 4 wherein the retinoid derivative is all trans-isomer, 9-cis-isomer, 11-cis-isomer, 13-cis-isomer, 9,11-di-cis-isomer, 9,13-di-cis-isomer, 11,13-di-cis-isomer, or 9,11,13-tri-cis-isomer.

6. The method of claim 4 wherein the positively-charged retinoid derivative is 11-cis retinylamine.

7. The method of claim 4 wherein the positively-charged retinoid derivative is 9-cis retinylamine, 13-cis retinylamine, or all trans retinylamine.

8. The method of claim 1, wherein the positively-charged retinoid derivative is a retinoid derivative of formula II: 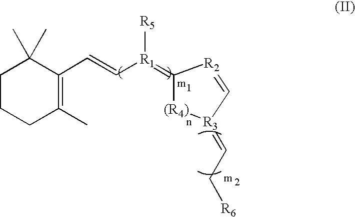
or a stereoisomer, prodrug, pharmaceutically or ophthamologically acceptable salt, hydrate, solvate, acid salt hydrate, N-oxide or isomorphic crystalline form thereof,
wherein n is 1, 2, 3, or 4;
m₁ plus m₂ equals 1, 2, or 3; and
wherein at least one of R₁, R₂, R₃, R₄, R₅ or R₆ is a primary, secondary, tertiary or quaternary amine;
wherein R₅ is, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₇R₈⁺X⁻, CH₂—NR₇R₈, NR₇R₈, or NR₇R₈R₉⁺X⁻;
wherein R₆ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₇R₈⁺X⁻, OR₇, SR₇, CH₂—NR₇R₈, NR₇R₉, or NR₇R₈R₉⁺X⁻;
wherein R₇ R₈, and R₉ are independently, H, C₁ to C₆ alkyl, C₂ to C₆ alkenyl, C₂ to C₆ alkynyl, or C₃ to C₄ cycloalkyl, OH, or OR₁₀, wherein R₁₀ is C₁ to C₆ alkyl; and X is an anion, Cl, Br, I, SO₃H, or P(O)₂(OH)₂.

9. The method of claim 1, wherein the positively-charged retinoid derivative is a retinoid derivative of formula III: 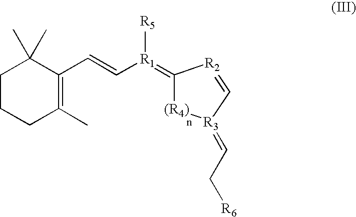
or a stereoisomer, prodrug, pharmaceutically or ophthamologically acceptable salt, hydrate, solvate, acid salt hydrate, N-oxide or isomorphic crystalline form thereof,
wherein n is 1, 2, 3, or 4; and
wherein at least one of R₁, R₂, R₃, R₄, R₅ or R₆ is a primary, secondary, tertiary or quaternary amine;
wherein R₅ is, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₇R₈⁺X⁻, CH₂—NR₇R₈, NR₇R₈, or NR₇R₈R₉⁺X⁻;
wherein R₆ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₇R₈⁺X⁻, OR₇, SR₇, CH₂—NR₇R₈, NR₇R₉, or NR₇R₈R₉⁺X⁻;
wherein R₇ R₈, and R₉ are independently, H, C₁ to C₆ alkyl, C₂ to C₆ alkenyl, C₂ to C₆ alkynyl, or C₃ to C₄ cycloalkyl, OH, or OR₁₀, wherein R₁₀ is C₁ to C₆ alkyl; and X is an anion, Cl, Br, I, SO₃H, or P(O)₂(OH)₂.

10. The method of claim 9 wherein the positively-charged retinoid derivative is 11-cis locked retinylamine.

11. The method of claim 1, wherein the positively-charged retinoid derivative is a retinoid derivative of formula IV: 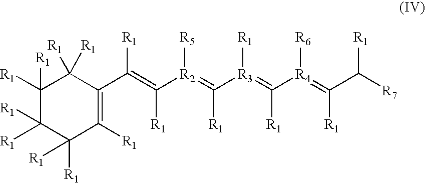
or a stereoisomer, prodrug, pharmaceutically or ophthamologically acceptable salt, hydrate, solvate, acid salt hydrate, N-oxide or isomorphic crystalline form thereof,
R₁ is, independently, hydrogen, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, OR₈, SR₈, or NR₈R₉, wherein R₈ and R₉ are, independently, H, C₁ to C₆ alkyl;
wherein at least one of R₂, R₃, R₄, R₅, R₆ or R₇ is a primary, secondary, tertiary or quaternary amine;
wherein R₅ or R₆ are, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₁₀R₁₁⁺X⁻, CH₂—NR₁₀R₁₁, NR₁₀R₁₁, or NR₁₀R₁₁, R₁₂⁺X⁻;
wherein R₇ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₁₀R₁₁⁺X⁻, OR₁₀, SR₁₀, CH₂—NR₁₀R₁₁, NR₁₀R₁₁, or NR₁₀R₁₁R₁₂⁺X⁻;
wherein R₁₀, R₁₁, and R₁₂ are independently, H, C₁ to C₆ alkyl, C₂ to C₆ alkenyl, C₂ to C₆ alkynyl, or C₃ to C₄ cycloalkyl, OH, or OR₁₃, wherein R₁₃ is C₁ to C₆ alkyl; and X is an anion, Cl, Br, I, SO₃H, or P(O)₂(OH)₂.

12. The method of claim 11, wherein the retinoid derivative is all trans-isomer, 9-cis-isomer, 11-cis-isomer, 13-cis-isomer, 9,11-di-cis-isomer, 9,13-di-cis-isomer, and 11,13-di-cis-isomer, or 9,11,13-tri-cis-isomer.

13. The method of claim 1, wherein the positively-charged retinoid derivative is a retinoid derivative of formula V: 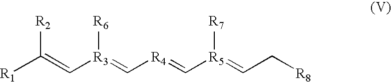
or a stereoisomer, prodrug, pharmaceutically or ophthamologically acceptable salt, hydrate, solvate, acid salt hydrate, N-oxide or isomorphic crystalline form thereof,
R₁ and R₂ are, independently, lower alkyl, straight chain alkyl, linear, iso-alkyl, sec-alkyl, tert-alkyl, C₁ to C₆ branched chain alkyl, substituted alkyl groups, substituted branched chain alkyl, hydroxyl, hydroalkyl, amine, or amide;
wherein at least one of R₃, R₄, R₅, R₆, R₇, or R₈ is a primary, secondary, tertiary or quaternary amine;
wherein R₆ or R₇ are, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₉R₁₀⁺X⁻, CH₂—NR₉R₁₀, NR₉R₁₀, or NR₉R₁₀R₁₁⁺X⁻;
wherein R₈ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₉R₁₀⁺X⁻, OR₇, SR₇, CH₂—NR₉R₁₀, NR₉R₁₀, or NR₉R₁₀R₁₁⁺X⁻;
wherein R₉ R₁₀, and R₁₁ are independently, H, C₁ to C₆ alkyl, C₂ to C₆ alkenyl, C₂ to C₆ alkynyl, or C₃ to C₄ cycloalkyl, OH, or OR₁₂, wherein R₁₂ is C₁ to C₆ alkyl; and X is an anion, Cl, Br, I, SO₃H, or P(O)₂(OH)₂.

14. The method of claim 1 wherein the degenerative disease is a result of lipofuscin pigment accumulation in the eye.

15. The method of claim 14 wherein the degenerative disease is a result of N-retinylidene-N-retinylethanolamine accumulation in the eye.

16. The method of claim 14 wherein the degenerative disease is age-related macular degeneration or Stargardt's macular dystrophy.

17. The method of claim 1, wherein the retinoid derivative is locally administered to the eye.

18. The method of claim 17, wherein the retinoid derivative is locally administered by eye drops, intraocular injection or periocular injection.

19. The method of claim 1, wherein the retinoid derivative is orally administered to the vertebrate.

20. A method for preventing photoreceptor degeneration in a vertebrate eye comprising administering to the vertebrate an effective amount of a positively charged retinoid compound in a pharmaceutically or ophthamologically acceptable vehicle, slowing chromophore flux in a retinoid cycle in the eye, and preventing photoreceptor degeneration in the eye.

21. The method of claim 20, wherein the positively charged retinoid compound is a retinylamine derivative.

22. The method of claim 20, wherein the positively charged retinoid compound inhibits an isomerization step of the retinoid cycle.

23. The method of claim 20, wherein the positively-charged retinoid compound is a retinoid derivative of formula I: 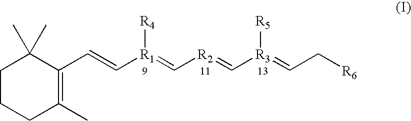
or a stereoisomer, prodrug, pharmaceutically or ophthamologically acceptable salt, hydrate, solvate, acid salt hydrate, N-oxide or isomorphic crystalline form thereof,
wherein at least one of R₁, R₂, R₃, R₄, R₅ or R₆ is a primary, secondary, tertiary or quaternary amine;
wherein R₄ or R₅ are, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₇R₈⁺X⁻, CH₂—NR₇R₈, NR₇R₈, or NR₇R₈R₉⁺X⁻;
wherein R₆ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₇R₈⁺X⁻, OR₇, SR₇, CH₂—NR₇R₈, NR₇R₈, or NR₇R₈R₉⁺X⁻;
wherein R₇, R₈, and R₉ are independently, H, C₁ to C₆ alkyl, C₂ to C₆ alkenyl, C₂ to C₆ alkynyl, or C₃ to C₄ cycloalkyl, OH, or OR₁₀ wherein R₁₀ is C₁ to C₆ alkyl; and X is an anion, Cl, Br, I, SO₃H, or P(O)₂(OH)₂.

24. The method of claim 23 wherein the positively-charged retinoid compound is 11-cis retinylamine.

25. The method of claim 23 wherein the positively-charged retinoid compound is all trans-isomer, 9-cis-isomer, 11-cis-isomer, 13-cis-isomer, 9,11-di-cis-isomer, 9,13-di-cis-isomer, 11,13-di-cis-isomer, or 9,11,13-tri-cis-isomer.

26. The method of claim 20, wherein the positively-charged retinoid compound is a retinoid derivative of formula II: 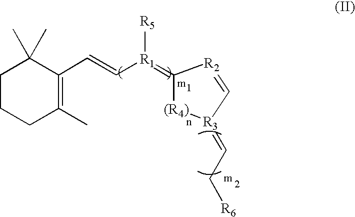
or a stereoisomer, prodrug, pharmaceutically or ophthamologically acceptable salt, hydrate, solvate, acid salt hydrate, N-oxide or isomorphic crystalline form thereof,
wherein n is 1, 2, 3, or 4;
m₁ plus m₂ equals 1, 2, or 3; and
wherein at least one of R₁, R₂, R₃, R₄, R₅ or R₆ is a primary, secondary, tertiary or quaternary amine;
wherein R₅ is, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₇R₈⁺X⁻, CH₂—NR₇R₈, NR₇R₈, or NR₇R₈R₉⁺X⁻;
wherein R₆ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₇R₈⁺X⁻, OR₇, SR₇, CH₂—NR₇R₈, NR₇R₈, or NR₇R₈R₉⁺X⁻;
wherein R₇, R₈, and R₉ are independently, H, C₁ to C₆ alkyl, C₂ to C₆ alkenyl, C₂ to C₆ alkynyl, or C₃ to C₄ cycloalkyl, OH, or OR₁₀, wherein R₁₀ is C₁ to C₆ alkyl; and X is an anion, Cl, Br, I, SO₃H, or P(O)₂(OH)₂.

27. The method of claim 20, wherein the positively-charged retinoid compound is a retinoid derivative of formula III: 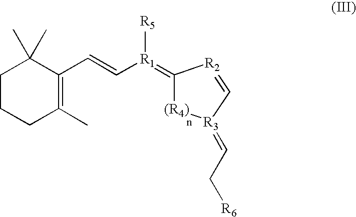
or a stereoisomer, prodrug, pharmaceutically or ophthamologically acceptable salt, hydrate, solvate, acid salt hydrate, N-oxide or isomorphic crystalline form thereof,
wherein n is 1, 2, 3, or 4; and
wherein at least one of R₁, R₂, R₃, R₄, R₅ or R₆ is a primary, secondary, tertiary or quaternary amine,
wherein R₅ is, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₇R₈⁺X⁻, CH₂—NR₇R₈, NR₇R₈, or NR₇R₈R₉⁺X⁻;
wherein R₆ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₇R₈⁺X⁻, OR₇, SR₇, CH₂—NR₇R₈, NR₇R₈, or NR₇R₈R₉⁺X⁻;
wherein R₇, R₈, and R₉ are independently, H, C₁ to C₆ alkyl, C₂ to C₆ alkenyl, C₂ to C₆ alkynyl, or C₃ to C₄ cycloalkyl, OH, or OR₁₀, wherein R₁₀ is C₁ to C₆ alkyl; and X is an anion, Cl, Br, I, SO₃H, or P(O)₂(OH)₂.

28. The method of claim 27 wherein the positively-charged retinoid compound is 11-cis locked retinylamine.

29. The method of claim 20, wherein the positively-charged retinoid compound is a retinoid derivative of formula IV: 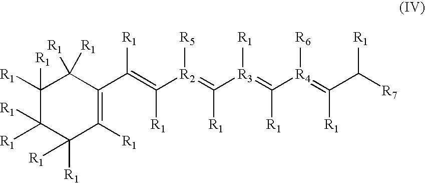
or a stereoisomer, prodrug, pharmaceutically or ophthamologically acceptable salt, hydrate, solvate, acid salt hydrate, N-oxide or isomorphic crystalline form thereof,
R₁ is, independently, hydrogen, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, OR₈, SR₈, or NR₈R₉, wherein R₈ and R₉ are, independently, H, C₁ to C₆ alkyl;
wherein at least one of R₂, R₃, R₄, R₅, R₆ or R₇ is a primary, secondary, tertiary or quaternary amine;
wherein R₅ or R₆ are, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₁₀R₁₁⁺X⁻, CH₂—NR₁₀R₁₁, NR₁₀R₁₁, or NR₁₀R₁₁R₁₂⁺X⁻;
wherein R₇ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridinium, pyrrolidinium, phosphonium, guanidinium, isouronium, iodonium, sulfonium, CH₂—SR₁₀R₁₁⁺X⁻, OR₁₀, SR₁₀, CH₂—NR₁₀R₁₁, NR₁₀R₁₁, or NR₁₀R₁₁, R₁₂⁺X⁻;
wherein R₁₀, R₁₁, and R₁₂ are independently, H, C₁ to C₆ alkyl, C₂ to C₆ alkenyl, C₂ to C₆ alkynyl, or C₃ to C₄ cycloalkyl, OH, or OR₁₃, wherein R₁₃ is C₁ to C₆ alkyl; and X is an anion, Cl, Br, I, SO₃H, or P(O)₂(OH)₂.

30. The method of claim 29, wherein the retinoid derivative is all trans-isomer, 9-cis-isomer, 11-cis-isomer, 13-cis-isomer, 9,11-di-cis-isomer, 9,13-di-cis-isomer, 11,13-di-cis-isomer, or 9,11,13-tri-cis-isomer.

31. The method of claim 20, wherein the positively-charged retinoid compound comprises a retinoid derivative of formula V: 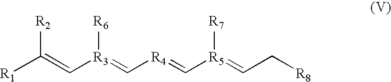
or a stereoisomer, prodrug, pharmaceutically or ophthamologically acceptable salt, hydrate, solvate, acid salt hydrate, N-oxide or isomorphic crystalline form thereof,
R₁ and R₂ are, independently, lower alkyl, straight chain alkyl, linear, iso-alkyl, sec-alkyl, tert-alkyl, C₁ to C₆ branched chain alkyl, substituted alkyl groups, substituted branched chain alkyl, hydroxyl, hydroalkyl, amine, or amide;
wherein at least one of R₃, R₄, R₅, R₆, R₇, or R₈ is a primary, secondary, tertiary or quaternary amine;
wherein R6 or R7 are, independently, H, C₁ to C₆ alkyl, C₂ to C₆ alkenyl, C₂ to C₆ alkynyl, or C₃ to C₄ cycloalkyl, disubstituted imidazolium, trisubstituted imidazolium, pyridinium, pyrrolidinium, phosphonium, guanidinium, isouronium, iodonium, sulfonium, CH₂—SR₉R₁₀⁺X⁻, CH₂—NR₉R₁₀, NR₉R₁₀, or NR₉R₁₀R₁₁⁺X⁻;
wherein R₈ is, independently, H, C₁ to C₁₄ alkyl, C₁ to C₁₄ alkenyl, C₁ to C₁₄ alkylyl, C₃ to C₁₄ branched alkyl, C₃ to C₁₀ cycloalkyl, halogen, heterocyclic, disubstituted imidazolium, trisubstituted imidazolium, pyridium, pyrrolidinium, phosphonium, guanidinium, isouronium, iodonium, sulfonium, CH₂—SR₉R₁₀⁺X⁻, OR₇, SR₇, CH₂—NR₉R₁₀, NR₉R₁₀, or NR₉R₁₀R₁₁⁺X⁻;
wherein R₉ R₁₀, and R₁₁ are independently, H, C₁ to C₆ alkyl, C₂ to C₆ alkenyl, C₂ to C₆ alkynyl, or C₃ to C₄ cycloalkyl, OH, or OR₁₂, wherein R₁₂ is C₁ to C₆ alkyl; and X is an anion, Cl, Br, I, SO₃H, or P(O)₂(OH)₂.

32. The method of claim 20 further comprising reducing accumulation of lipofuscin pigment in the eye.

33. The method of claim 32 wherein the lipofuscin pigment is N-retinylidene-N-retinylethanolamine.

34. The method of claim 32, wherein reducing accumulation of lipofuscin pigment in the eye is a treatment for degenerative eye disease, age-related macular degeneration, or Stargardt's macular dystrophy.

35. The method of claim 20, wherein the retinoid compound is locally administered to the eye.

36. The method of claim 35, wherein the synthetic retinoid is locally administered by eye drops, intraocular injection or periocular injection.

37. The method of claim 20, wherein the synthetic retinoid is orally administered to the vertebrate.
